SEQUENCE LISTING (15 sequences)

SEQ ID NO: 1 - protein, 7 aa, Artificial Sequence
  Peptides have been prepared using Boc or Fmoc chemistry and solid phase peptide synthesis
XKKKRKV

SEQ ID NO: 2 - protein, 14 aa, Artificial Sequence
  Peptides have been prepared using Boc or Fmoc chemistry and solid phase peptide synthesis
QELQSIEXELQAIE

SEQ ID NO: 3 - protein, 22 aa, Artificial Sequence
  Peptides have been prepared using Boc or Fmoc chemistry and solid phase peptide synthesis
XEIAQLEYEISQLEQEIQALES

SEQ ID NO: 4 - protein, 16 aa, Artificial Sequence
  Peptides have been prepared using Boc or Fmoc chemistry and solid phase peptide synthesis
XQELQSIEYELQAIEX

SEQ ID NO: 5 - protein, 23 aa, Artificial Sequence
  Peptides have been prepared using Boc or Fmoc chemistry and solid phase peptide synthesis
XSELAQIEQELQSIEYELQAIEX

SEQ ID NO: 6 - protein, 9 aa, Artificial sequence
  Peptides have been prepared using Boc or Fmoc chemistry and solid phase peptide synthesis
XEEEEGGGS

SEQ ID NO: 7 - protein, 10 aa, Artificial sequence
  Peptides have been prepared using Boc or Fmoc chemistry and solid phase peptide synthesis
XKEEEEGGGS

SEQ ID NO: 8 - protein, 17 aa, Artificial sequence
  Peptides have been prepared using Boc or Fmoc chemistry and solid phase peptide synthesis
XRQIKIWFQNRRMKWKX

SEQ ID NO: 9 - protein, 10 aa, Artificial sequence
  Peptides have been prepared using Boc or Fmoc chemistry and solid phase peptide synthesis
XEEEEGGGSC

SEQ ID NO: 10 - protein, 10 aa, Artificial sequence
  Peptides have been prepared using Boc or Fmoc chemistry and solid phase peptide synthesis
XKPKKKRKVC

SEQ ID NO: 11 - protein, 11 aa, Artificial sequence
  Peptides have been prepared using Boc or Fmoc chemistry and solid phase peptide synthesis
XKPKKKRKVXK

SEQ ID NO: 12 - protein, 20 aa, Artificial sequence
  Peptides have been prepared using Boc or Fmoc chemistry and solid phase peptide synthesis
XKKKKKKKKKKKPKKKRKVC

SEQ ID NO: 13 - protein, 10 aa, Artificial sequence
  Peptides have been prepared using Boc or Fmoc chemistry and solid phase peptide synthesis
XKKKKKKKKK

SEQ ID NO: 14 - protein, 9 aa, Artificial sequence
  Peptides have been prepared using Boc or Fmoc chemistry and solid phase peptide synthesis
PKKKKKKKK

SEQ ID NO: 15 - protein, 7 aa, Artificial sequence
  Peptides have been prepared using Boc or Fmoc chemistry and solid phase peptide synthesis
PKKKRKV

(19) United States
(12) Patent Application Publication
(10) Pub. No.: US 2012/0034162 A1
(43) Pub. Date: Feb. 9, 2012

(54) Fullerene Assisted Cell Penetrating Peptides

(75) Inventors: Andrew R. Barron, Houston, TX (US); Jianzhong Yang, Missouri City, TX (US); Jianhua Yang, Bellaire, TX (US); Kuan Wang, Heilongjiang (CN); Jonathan Driver, Katy, TX (US)

(73) Assignee: William March Rice University, Houston, TX (US)

(21) Appl. No.: 12/294,991

(22) PCT Filed: Mar. 30, 2007
(86) PCT No.: PCT/US2007/065654

§ 371 Date: Jul. 15, 2011

Related U.S. Application Data
(60) Provisional application No. 60/787,954, filed on Mar. 31, 2006.

(51) Int. Cl. A61K 51/08; C07K 19/00; C12N 5/071; A61K 38/02; A61K 38/14; A61K 9/127; C07K 2/00; C07K 1/107; B82Y 5/00

(52) U.S. Cl. 424/1.69; 530/300; 530/322; 530/409; 435/375; 530/334; 514/1.1; 514/20.9; 424/450; 977/773; 977/915; 977/738

(57) ABSTRACT

A composition and method is described for intracellular delivery of fullerene containing peptides. The composition and method involve fullerene-substituted phenylalanine as part of a peptide based delivery system. The presence of a fullerene-substituted amino acid in a peptide is found to alter the intracellular transport properties of the peptide.

### CROSS-REFERENCE TO RELATED APPLICATIONS

This application claims priority under Title 35 United States Code, §119 to U.S. provisional patent application U.S. Pat. App. Ser. No. 60/787,954 filed Mar. 31, 2006.

### FIELD OF THE INVENTION

The field of the invention relates generally to intracellular delivery.

### BACKGROUND OF THE INVENTION

Efficient intracellular delivery of a drug can not only reduce non-specific effects and toxicity, allowing for lower dosage levels with a concomitant decrease in side effects, but also enhance the effectiveness of drugs incapable of reaching their in vivo therapeutic target. Cell penetrating peptides (CPPs) have drawn widespread attention as potential drug delivery agents over the past decade. P. Lundberg, Ü. Langel, J. Mol. Recognit. 16, 227-233 (2003). J. P. Richard, K. Melikov, E. Vives, C. Ramos, B. Verbeure, M. J. Gait, L. V. Chemomordik, B. Lebleu, J. Biol. Chem. 278, 585-590 (2003). C. Foerg, U. Ziegler, J. Fernandez-Carneado, E. Giral, R. Rennert, A. G. Beck-Sickinger, H. P. Merkle, Biochemistry 44, 72-81 (2005). Some simple examples are oligocationic peptides, e.g., Tat3, pentratin4, and oligoarginine. These are derived from short peptides of the protein transduction domains of virus proteins, and cannot only be internalized into cells, but also can deliver conjugated species into the cell membrane. In this way many species of biological importance have been delivered, including antigenetic peptides, peptide nucleic acids, antisense oligonucleotides, and proteins. Since a major limitation in developing peptide- and nucleic acid-based drugs is their inability to enter the cell, the conjugation of therapeutic agents to CPPs has become a strategy of choice to improve their pharmacological properties.

Transport of any species into the nucleus of an intact cell is limited by at least three major membrane barriers, namely the cell membrane, the endosomal membrane, and the nuclear membrane. It has been reported that condensed aromatic rings and anionic fullerenes could enhance cellular uptake of a simple CPP [octaarginine (R₈)] through a counter-anion-mediated oligo/polyarginine activation process. F. Perret, M. Nishihara, T. Takeuchi, S. Futaki, A. N. Lazar, A. W. Coleman, N. Sakai, S. Matile, J. Am. Chem. Soc. 127, 1114-1115 (2005).

Several approaches have been taken towards fullerene-based amino acids. The simplest approaches involve the reaction of an amino acid with C₆₀; however, in these derivatives only the carboxylic acid functional group is available for reaction, limiting subsequent incorporation into peptides. Truly bi-functional fullerene substituted amino acids (those in which both the carboxylic acid and amine functionalities are available for reaction) have been limited to those employing ester or amide linkages. M. Maggini, G. Scorrano, A. Bianco, C. Toniolo, R. P. Sijbesma, F. Wudl, M. Prato, Chem. Commun. 305-306 (1994). A. Skiebe, A. Hirsch, Chem. Commun. 335-338 (1994). Fullerene peptides have shown potential applications in medicinal chemistry, D. Pantarotto, N. Tagmatarchis, A. Bianco, M. Prato Mini-Reviews in Medicinal Chemistry 4, 805-814 (2004), however, fullerene amino acids suitable for solid phase peptide synthesis (SPPS) have been of limited success due to the instability of the ester or amide linkages.

Thus, there is a continuing need for the development of bi-functional fullerene based amino acids that form stable linkages and compatible solid phase synthetic methods.

### BRIEF SUMMARY OF THE INVENTION

In one aspect, the present invention is a cell penetrating peptide for intracellular delivery comprising a fullerene modified amino acid and a peptide, wherein the cell penetrating peptide is capable of crossing a cell membrane. In some embodiments, the fullerene modified amino acid may be selected from the group including but not limited to a fullerene substituted phenylalanine derivative, a fullerene substituted lysine derivative, a fullerene substituted C₇₀ derivative and a metallo-fullerene derivative. In some embodiments, the fullerene substituted phenylalanine derivative may be attached to the peptide by reaction with a Boc-derivatized amino acid or a Fmoc-derivatized amino acid. In some embodiments, the cell penetrating peptide is selected from the group including but not limited to a cationic peptide, a neutral peptide and a anionic peptide. In some embodiments, the cell penetrating peptide may contain a fluorescent label. In some embodiments, the cell penetrating peptide may be a targeting peptide, such as a nuclear localization sequence. In some embodiments, the cell penetrating peptide is delivered to the cytoplasm. In some embodiments, the cell penetrating peptide may further comprise an entity selected from the group consisting of drug species, diagnostic probes, antigenetic peptides, peptide nucleic acids, antisense oligonucleotides, proteins, nanoparticles, liposomes and radioactive material.

Another embodiment of the present invention is a method of intracellular delivery comprising: (a) obtaining a cell penetrating peptide comprising a fullerene modified amino acid and (b) incubating the cell penetrating peptide with cells. In some embodiments, the cell penetrating peptide may be targeted to a specific area within the cell.

Yet another embodiment of the present invention is a method of synthesizing a cell penetrating peptide comprising: (a) synthesizing a peptide using solid phase peptide synthesis and (b) coupling a fullerene to the peptide to form a cell penetrating fullerene peptide. In some embodiments, the fullerene may be a fullerene modified amino acid. In some embodiments, the fullerene modified amino acid may be selected from the group including but not limited to a fullerene substituted phenylalanine derivative, a fullerene substituted lysine derivative, a C₇₀ fullerene derivative and a metallo-fullerene derivative. In some embodiments, the fullerene substituted phenylalanine derivative may be attached to the peptide by reaction with a Boc-derivatized amino acid or a Fmoc-derivatized amino acid. In some embodiments, the method may further comprise reacting the peptide with a fluorescent label.

Still another embodiment of the present invention is a method of treatment comprising: obtaining a cell penetrating fullerene peptide and administering to a patient a cell penetrating fullerene peptide. In some embodiments, the cell penetrating fullerene peptide is designed to target a specific function of cell growth. In some embodiments, the cell penetrating fullerene peptide is targeted based on the patient's DNA. In some embodiments, the cell penetrating fullerene peptide may deliver an entity selected from the group consisting of drug species, diagnostic probes, antigenetic peptides, peptide nucleic acids, antisense oligonucleotides, proteins, nanoparticles, liposomes and radioactive material.

The foregoing has outlined rather broadly the features and technical advantages of a number of embodiments of the present invention in order that the detailed description of the present invention that follows may be better understood. Additional features and advantages of the invention will be described hereinafter.

### BRIEF DESCRIPTION OF THE DRAWINGS

The foregoing summary as well as the following detailed description of the preferred embodiment of the invention will be better understood when read in conjunction with the appended drawings. It should be understood, however, that the invention is not limited to the precise arrangements and instrumentalities shown herein. The components in the drawings are not necessarily to scale, emphasis instead being placed upon clearly illustrating the principles of the present invention.

The invention may take physical form in certain parts and arrangement of parts. For a more complete understanding of the present invention, and the advantages thereof, reference is now made to the following descriptions taken in conjunction with the accompanying drawings, in which:

FIG. 1. shows a Bucky (C60) amino acid (Baa) derivative of phenylalanine and its Boc protected form.

FIG. 2. shows a Baa derivative of lysine and its Boc protected form.

FIG. 3. shows a C70 amino acid derivative of phenylalanine and its Boc protected form.

FIG. 4. shows a fullerene peptide with a FITC label.

FIG. 5. shows another fullerene peptide with a FITC label.

FIG. 6. (a) HPLC chromatogram of Baa-Lys(FITC)-(Lys)₈-OH (b) MALDI-ToF MS of Baa-Lys(FITC)-(Lys)₈-OH (c) UV-visible absorption spectrum of Baa-Lys(FITC)-NLS (d) fluorescence spectrum of Baa-Lys(FITC)-NLS.

FIG. 7. shows the aggregation of a FITC labeled fullerene peptide 25 as a function of concentration.

FIG. 8. shows the aggregation of another FITC labeled fullerene peptide 26 as a function of concentration.

FIG. 9. shows cyro TEM micrograph of (a) large aggregates and (b) small aggregates of a FITC labeled fullerene peptide 25.

FIG. 10. shows cyro TEM micrograph of (a) large aggregates and (b) small aggregates of a FITC labeled fullerene peptide 26.

FIG. 12. shows a CD spectrum of fullerene peptide 21.

FIG. 13. shows TEM images of (a) fibrous forms of fullerene peptide 21 and (b) micellar forms of fullerene peptide 21.

FIG. 14. shows a CD spectrum of fullerene peptide 22.

FIG. 15. shows a TEM image of peptide 22.

FIG. 16. shows a CD spectrum of fullerene peptide 23.

FIG. 17. shows a TEM image of peptide 23.

FIG. 18. shows the inhibitory activity of fullerene peptide 28 as a function of concentration.

FIG. 19. shows optical micrographs of HEK-293a cells incubated with (a) Lys(FITC)-(Lys)₈-OH and (b) Baa-Lys(FITC)-(Lys)₈-OH, and fluorescence images of HEK-293a cells incubated with (c) Lys(FITC)-(Lys)₈-OH, (d) Baa-Lys(FITC)-(Lys)₈-OH, (e) Phe-Lys(FITC)-NLS, and (f) Baa-Lys(FITC)-NLS.

FIG. 20. shows fluorescence micrographs of neuroblastoma cells incubated with (green) Baa-Lys(FITC)-NLS and (blue) DAPI. Scale bar=150 μm.

FIG. 21. shows MTT viability test of Baa for 24 hrs incubation and 48 hrs incubation from 0.00004 to 0.4 mg/mL.

FIG. 22 shows a TEM image of HEK cells treated with 0.4 mg·mL-1 BAA for 24 hrs.

FIG. 23 shows MTT viability of H-Baa-Lys(FITC)-NLS from 0.001 to 0.4 mg/mL

FIG. 24 shows inhibition of breast cancer cell line (MCF-7) with fullerene peptides.

FIG. 25 shows inhibition of neuroblastoma cancer cell line (MDA-MB) with fullerene peptides.

FIG. 26 shows inhibition of neuroblastoma cancer cell line (IMR32) with fullerene peptides.

### DETAILED DESCRIPTION OF THE INVENTION

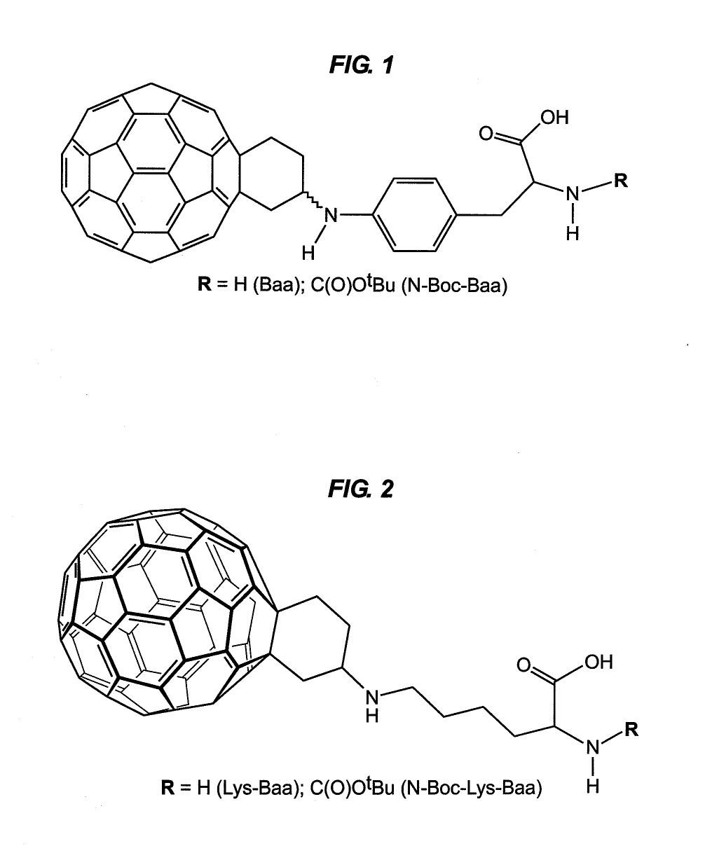

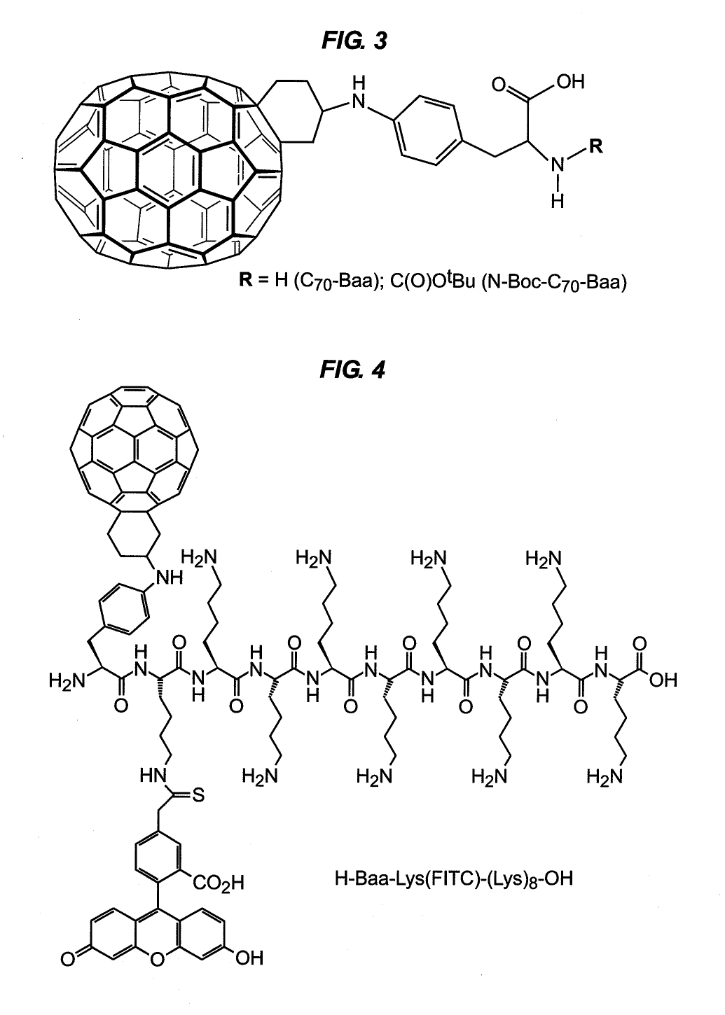

Intracellular drug delivery and targeted diagnostic probe delivery are important in drug development, disease diagnosis and disease treatment. A new approach to intracellular delivery has been developed using peptides containing fullerene modified amino acids. A fullerene substituted phenylalanine derivative (Bucky amino acid, Baa), J. Yang, A. R. Barron. Chem. Commun. 2884-2886 (2004), (100, FIG. 1) has been used as part of a peptide based drug system. Furthermore, a lysine derivative can be used (FIG. 2). Additionally, amino acids based upon higher fullerenes such as C₇₀ are equally useful (FIG. 3) as are those derived from metallo-fullerenes.

Baa is a hydrolytically stable fullerene amino acid. The hydrolytic stability of the fullerene substituted amino acid allows for peptides to be synthesized by SPPS where the presence of a fullerene-based amino acid is found to alter the intracellular transport properties of the peptide. The fullerene acts as a passport for intracellular delivery allowing the transport of cationic peptides into cells, including but not limited to human HEK-293 cells. The peptides in the absence of the fullerene amino acid cannot enter the cell. Similar results may be obtained with alternative cell types, for example, the human liver cancer cell line (HepG2) and the neuroblastoma cell line (IMR 32). Specific delivery of the fullerene species into either the cytoplasm or nucleus of the cell is also demonstrated. The nuclear localization signal (NLS) fullerene peptide, H-Baa-Lys(FITC)-Lys-Lys-Arg-Lys-Val-OH, can actively cross the cell membrane and accumulate significantly in the nucleus of HEK-293 cells, while H-Baa-Lys(FITC)-Lys₈-OH accumulates in the cytoplasm. Examples of other fullerene peptides include but are not limited to: Glu-Ile-Ala-Gln-Leu-Glu-Baa-Glu-Ile-Ser-Gln-Leu-Glu-Gln-NH₂, Baa-Glu-Ile-Ala-Gln-Leu-Glu-Tyr-Glu-Ile-Ser-Gln-Leu-Glu-Gln-NH₂ (Baa-2HP), Baa-Glu-Ile-Ala-Gln-Leu-Glu-Tyr-Glu-Ile-Ser-Gln-Leu-Glu-Gln-Glu-Ile-Gln-Ala-Leu-Glu-Ser-NH₂ (Baa-3HP), Baa-(Lys)₈-OH, Baa-Glu-Glu-Glu-Glu-Gly-Gly-Gly-Ser-OH, Baa-Lys(FITC)-Glu-Glu-Glu-Glu-Gly-Gly-Gly-Ser-OH, Baa-Arg-Gln-Ile-Lys-Ile-Trp-Phe-Gln-Asn-Arg-Arg-Met-Lys-Trp-Lys-Lys-OH, Baa-Glu-Glu-Glu-Glu-Gly-Gly-Gly-Ser-Cys-OH, Baa-Lys(FITC)-Pro-Lys-Lys-Lys-Arg-Lys-Val-Cys-OH, Baa-(Lys)₁₀-Lys(FITC)-Pro-Lys-Lys-Lys-Arg-Lys-Val-Cys-OH. Alternatively, the peptide sequence can be chosen so as to mimic a portion of a desired protein sequence. The addition of the fullerene amino acid also facilitates delivery of anionic peptides into the cytoplasm, but at a lower efficiency than that of cationic peptides. Finally, peptide sequences may be constructed that include the amino acid cysteine in order to facilitate attachment of targeting molecules such as antibodies.

The addition of fullerene-based amino acids to cationic peptides facilitates their intercellular translocation into cells where the parent peptides (the peptide sequence without a fullerene modified amino acid attached) is not translocated. In this regard, the fullerene provides a passport for the peptide sequence for transport across the cell membrane. The combination of the hydrophobic C₆₀ core plus the hydrophilic peptide sequence may act as an amphipathic cell penetrating peptide. This concept is a new approach for overcoming the barrier for the effective delivery of membrane impermeable molecules. The Baa residue is relatively small, stable under physiological conditions, and readily added to any sequence. Fullerene amino acid containing cationic peptides have potential as a family of nanovectors for targeted drug delivery.

The fullerene peptides are effective against various cancer cell lines and have activity with a wide range of cell types. These compounds have been characterized by methods including IR, UV, HPLC and MS. The new peptides, which are additions to the fullerene amino acid residue family, may possess potential pharmaceutical applications and may provide a new platform for further exploration in cancer therapy, targeted drug delivery and peptide and protein engineering.

The present invention provides a stable fullerene-based amino acid, such as Baa, and adds it at the end of a peptide sequence. The peptide sequence alone would not ordinarily transport across the cell membrane. However, the peptide is chosen to specifically act with a portion of the cell. For example, it can be chosen to bind specifically with the cell nucleus. The combination of the fullerene and peptide allow the peptide to be transported into the cell through the membrane and targeted to the desired point.

Fullerene peptides may be synthesized through a number of routes. One route is the solid phase coupling of Boc-Baa with different peptide sequences on a resin. A second route to obtain the desired peptide sequence is through the coupling of Fmoc-Baa with different peptide sequences on a resin. In addition, the Boc and Fmoc derivatives of other fullerene amino acids are possible. Peptides have been prepared using Boc or Fmoc chemistry and solid phase peptide synthesis.

The fullerene peptides possess the unique ability to cross the cell membrane. Therefore, the fullerene peptides can act as nanovectors to deliver entities selected from the list including but not limited to, drug species, diagnostic probes, antigenetic peptides, peptide nucleic acid, antisense oligonucleotides, proteins, and even nanoparticles, liposomes, and radioactive material, into cell and cell nucleus through a covalent or non-covalent route. Given the unique shape and properties of fullerene materials, a fullerene peptide could function as a vehicle for drug or radioactive delivery in cancer therapy. Specifically designed fullerene peptides could be targeted to specific functions in cell growth for use in cancer therapy. The fullerene peptides could also be targeted based upon an individual's DNA.

The area of the cell to which the entity is delivered is dependent upon the peptide selected. The entity is carried by the cell penetrating fullerene peptide by methods including conjugation to the fullerene peptide and inclusion within the fullerene. For example, metallo-fullerenes contain a metal atom or atoms within the fullerene cage. These metals may be chosen from a wide range including metals that are radioactive or are suitable as MRI contrast agents. Conjugation to the fullerene may either be through covalent attachment, hydrogen bonding to the peptide sequence or by van der Waal forces. For example, cyclodextrins are known to encapsulate fullerenes through van der Waal forces.

### EXAMPLES

The following examples are provided to more fully illustrate some of the embodiments of the present invention. It should be appreciated by those of skill in the art that the techniques disclosed in the examples which follow represent techniques discovered by the inventors to function well in the practice of the invention, and thus can be considered to constitute exemplary modes for its practice. However, those of skill in the art should, in light of the present disclosure, appreciate that many changes can be made in the specific embodiments that are disclosed and still obtain a like or similar result without departing from the spirit and scope of the invention.

### Peptide Selection

Two cationic peptides were investigated. A polylysine derivative (primary sequence H-Pro-Lys-Lys-Lys-Lys-Lys-Lys-Lys-Lys-OH) was chosen because it is known that oligolysines are not cell penetrating peptides. In addition, once inside a cell, the oligolysine would show no specific targeting propensity and would show general uptake in the cytoplasm.

To demonstrate selective uptake, the SV-40 T antigen nuclear localization sequence (NLS; primary sequence H-Pro-Lys-Lys-Lys-Arg-Lys-Val-OH) was chosen. This heptapeptide serves as an “address label” for proteins, and leads to their targeting of the cell nucleus. The NLS peptide has to be located in the cytoplasm to achieve this goal. The NLS peptide is not readily incorporated into cells. Even if endosomal uptake did occur, the peptide or its conjugate may not be able to be released into cytoplasm and eventually would be excluded from the cells again.

The fullerene peptide derivatives were prepared and compared to their non-fullerene containing parent peptide. Table 1 summarizes exemplary Baa-containing peptides studied in accordance with embodiments disclosed herein.

TABLE 1

| Peptide | Sequence |
|---|---|
| 21 | Glu-Ile-Ala-Gln-Leu-Glu-Baa-Glu-Ile-Ser-Gln-Leu-Glu-Gln-NH₂ |
| 22 | Baa-Glu-Ile-Ala-Gln-Leu-Glu-Tyr-Glu-Ile-Ser-Gln-leu-Glu-Gln-NH₂(Baa-2HP) |
| 23 | Baa-Glu-Ile-Ala-Gln-Leu-Glu-Tyr-Glu-Ile-Ser-Gln-Leu-Glu-Gln-Glu-Ile-Gln- |
| | Ala-Leu-Glu-Ser-NH₂(Baa-3HP) |
| 24 | Baa-(Lys)₈-OH |
| 25 | Baa-Lys(FITC)-(Lys)₈-OH |
| 26 | Baa-Lys(FITC)-Pro-Lys-Lys-Lys-Arg-Lys-Val-OH(Baa-Lys(FITC)-NLS) |
| 27 | Baa-Glu-Glu-Glu-Glu-Gly-Gly-Gly-Ser-OH |
| 28 | Baa-Lys(FITC)-Glu-Glu-Glu-Glu-Gly-Gly-Gly-Ser-OH |
| 29 | Baa-Arg-Gln-Ile-Lys-Ile-Trp-Phe-Gln-Asn-Arg-Arg-Met-Lys-Trp-Lys-Lys-OH |
| | (Baa-Penetratin) |
| 210 | Baa-Glu-Glu-Glu-Glu-Gly-Gly-Gly-Ser-Cys-OH |
| 211 | Baa-Lys(FITC)-Pro-Lys-Lys-Lys-Arg-Lys-Val-Cys-OH |
| 212 | Baa-(Lys)₁₀-Lys(FITC)-Pro-Lys-Lys-Lys-Arg-Lys-Val-Cys-OH |

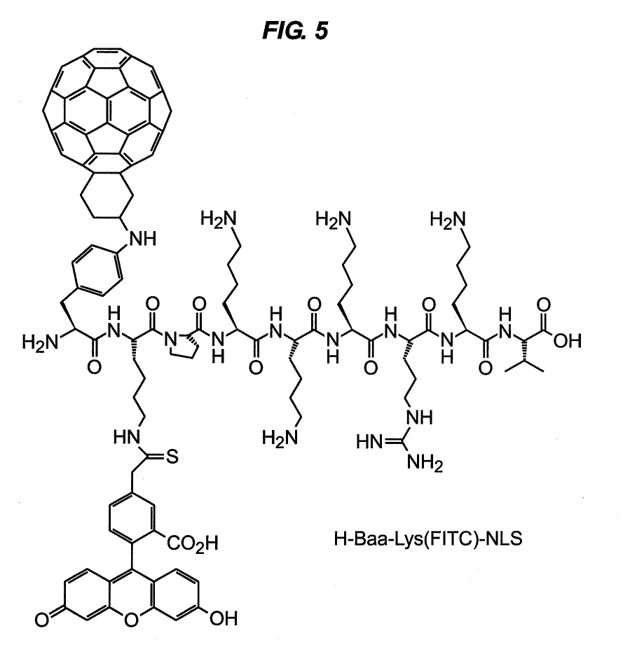

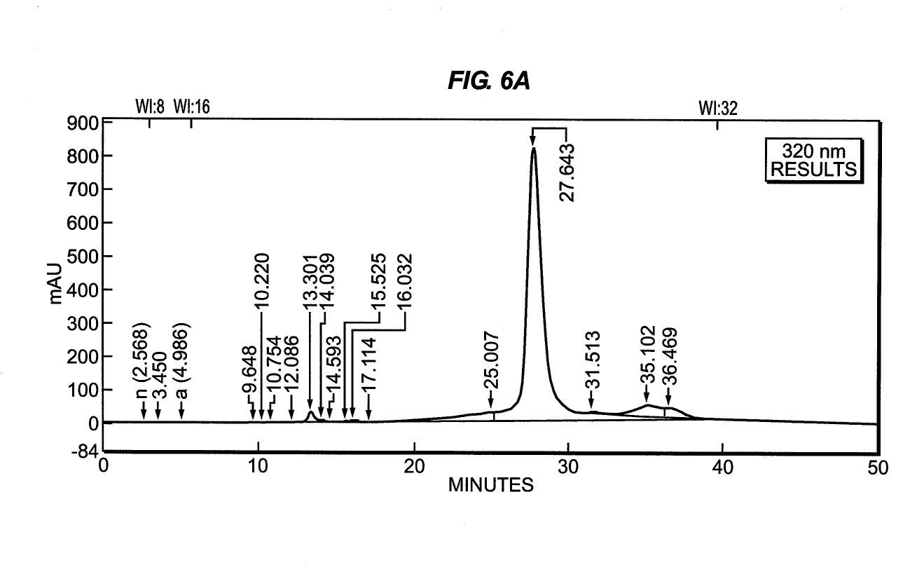

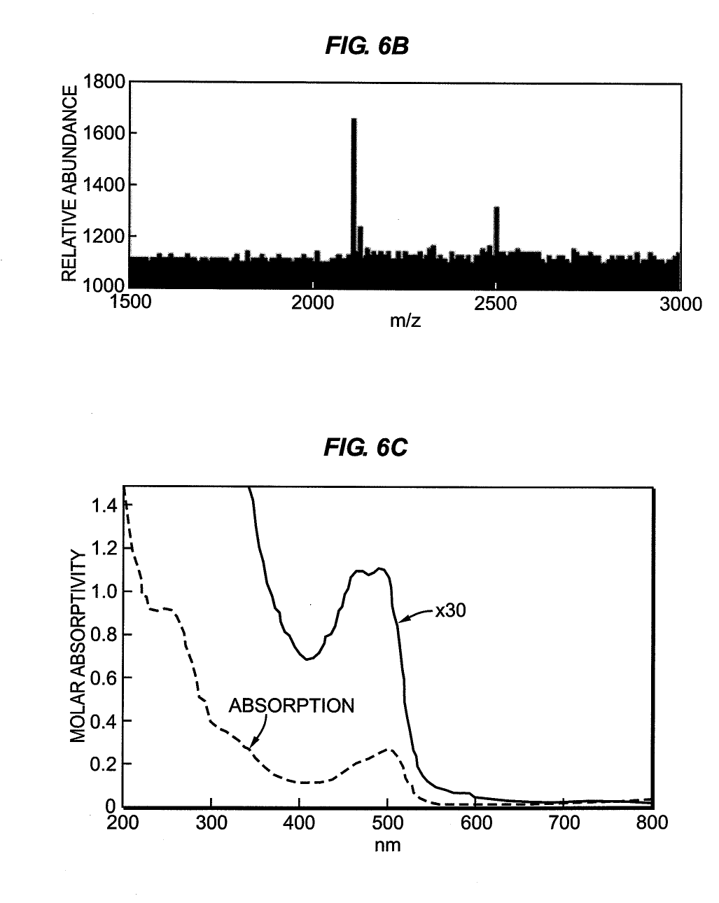

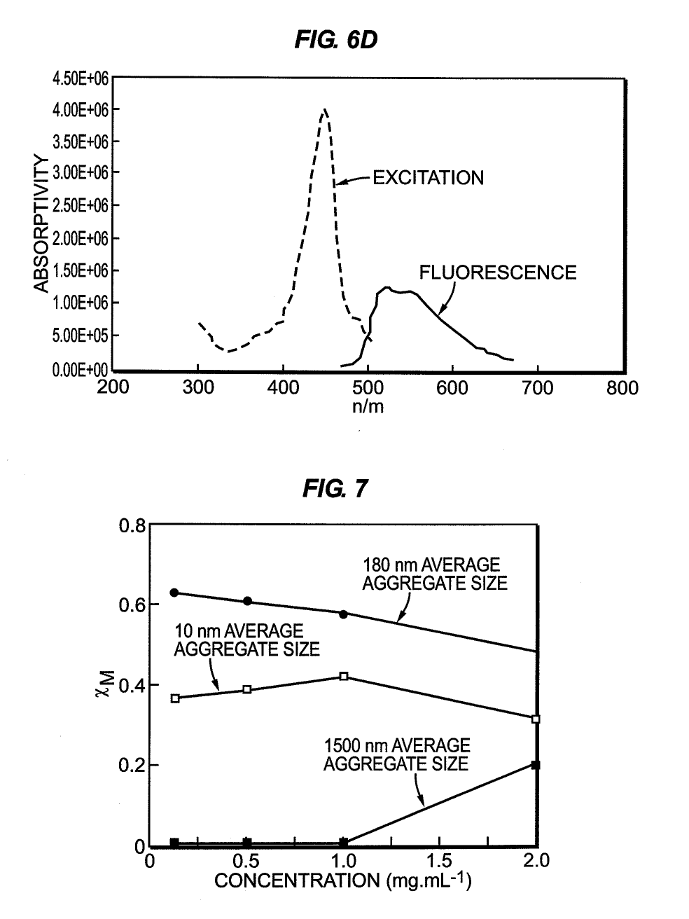

To visualize the fullerene peptide inside the cells, fluorescein isothiocyanate (FITC) was introduced as a label. Thus, the Baa derivatives (H-Baa-Lys(FITC)-Lys₈-OH (FIG. 4) and H-Baa-Lys(FITC)-NLS (FIG. 5) as well as the associated parent sequences (H-Lys(FITC)-Lys₈-OH and H-Phe-Lys(FITC)-NLS) were prepared by solid phased peptide synthesis (SPPS) on preloaded Wang resin. In the case of the NLS sequence, a phenylalanine was coupled to the end to mimic the phenylalanine substructure of the Baa amino acid residue.

Rink amide and Wang resin were obtained from Novabiochem, USA. Amino acids were purchased from Novabiochem and used as received. MALDI-TOF mass analysis was performed on a linear Protein-TOF Bruker instrument using sinipinic acid as the matrix. CD spectra were obtained on a Jasco J-700 dichrometer using 1 mm path-length quartz cells. Peptide solutions were 1.0 mg·mL⁻¹ solution in milliQ H₂O or PBS buffer. In the case of peptides (21, 22 and 23), the pH was first adjusted to 10 to break up any aggregations and then adjusted by addition of 0.1 mM HCl until the desired pH was obtained. CD spectra were recorded in millidegrees and converted to residual molar ellipticity. TEM measurements were performed on a JEOL 2010 TEM at 200 kV. DLS Measurements were performed on the samples using an Brookhaven 90Plus submicron particle-size analyzer with HeNe laser (30 mW) that operates at 656 nm wavelength.

### Example 1

Glu-Ile-Ala-Gln-Leu-Glu-Baa-Glu-Ile-Ser-Gln-Leu-Glu-Gln-NH₂ (21). The coupling of the first 6 residues was carried out on an automated APEX 396 Multiple Peptide Synthesizer (Advanced ChemTech) under nitrogen flow using a rink amide resin (430 mg, 0.3 mM) as the solid phase. Each coupling uses four-fold excess of the amino acid, and HBTU, HOBt as activators and DIEA as base in a 1:1:1:3 ratio. Fmoc deprotection was performed using 25% piperidine in DMF solution. After the deprotection of the sixth residue (Glu), one sixth of the resin (ca. 0.05 mM) was placed in a 25 mL fritted glass tube, and swollen with DMF (ca. 10 mL). A 3-fold excess of Fmoc-Baa was dissolved in DMF/DCM (2:1) (9 mL) in a second glass vial. The Fmoc-Baa solution was first activated with PyBOP/HOBt/DIEA (1:1:1:2) for 2 minutes, then mixed with the resin in the fritted glass tube, and shaken on an automated shaker for 1 day at room temperature. Then the resin was washed thoroughly with DMF and DCM to remove unreacted Fmoc-Baa, and retransferred in the automated synthesizer reactor. Fmoc removal was performed by the synthesizer using a 5% DBU solution in DMF. The subsequent amino acid couplings were accomplished using the conditions described above. The final peptide was cleaved from the solid support by washing with TFA:TIPS:H₂O (98:1:1) (10 mL) for 4 h and a second time for 18 hrs. The crude fractions were washed with Et₂O and lyophilized to remove TFA. Purification was carried out on an Varian C4 column using a gradient of TFA (0.1%) in H₂O to TFA in IPA (0.1%) over 75 min at flow rate of 5.0 mL·min⁻¹. The elution time was 57 min. Yield: 8.5 mg (6.8%). MALDI-MS: m/z, calculated 2488 (M⁺+Na). Found 2489.

### Example 2

Baa-Glu-Ile-Ala-Gln-Leu-Glu-Tyr-Glu-Ile-Ser-Gln-Leu-Glu-Gln-NH₂ (22). The solid phase synthesis of fullero-peptide 22 was carried out on an automated APEX 396 Multiple Peptide Synthesizer (Advanced ChemTech) under nitrogen flow. Rink amide resin (430 mg, 0.3 mM) was used as solid phase. Each coupling uses 4 fold amino acid excess, and HBTU, HOBt as activators and DIEA as base in a 1:1:1:3 ratio. Fmoc deprotection was performed using 25% piperidine in DMF solution. After the deprotection of the eighth residue (Glu) was finished, one sixth of the resin (ca. 0.05 mM) was moved out to a 25 mL flitted glass tube, swollen with DMF and a 3-fold excess of BocBaa (157 mg, 0.15 mmol) was dissolved in DMF/DCM (2:1, 9 mL). The Boc Baa solution was first activated with PyBOP/HOBt/DIEA (1:1:1:3) for 2 minutes. The activated Boc-Baa was mixed with the resin in the flitted glass tube, and shaken on an automated shaker for 1 day at room temperature. Then the resin was washed thoroughly with DMF and DCM to remove unreacted BocBaa. The amine linkage on Baa was acetylated by acetic acid anhydride (0.3 mL, ×2) for 4 hrs. The final peptide was cleaved twice from the solid support using 10 mL TFA:TIPS:H₂O (95:2.5:2.5) for 4 h and 18 hr. The crude fraction were washed with Et₂O and lyophilized to remove TFA. RP-HPLC purification was carried out on a Phenomenex Luna C5 column using an isocratic gradient of A: 0.1% TFA in water, and B: 0.1% TFA in isopropanol, 70% B, at 5.0 mL/min flow rate. The elution time was 41 min. After purification 20.6 mg (15.4%) were recovered. MALDI-MS: m/z calculated 2671 [M++Na]. Found 2671.

### Example 3

Baa-Glu-Ile-Ala-Gln-Leu-Glu-Tyr-Glu-Ile-Ser-Gln-Leu-Glu-Gln-Glu-Ile-Gln-Ala-Leu-Glu-Ser-NH₂ (23). The solid phase synthesis of fullero-peptide 23 was carried out on an automated APEX 396 Multiple Peptide Synthesizer (Advanced ChemTech) under nitrogen flow. Rink amide resin (430 mg, 0.3 mM) was used as solid phase. Each coupling uses 4 fold amino acid excess, and HBTU, HOBt as activators and DIEA as base in a 1:1:1:3 ratio. Fmoc deprotection was performed using 25% piperidine in DMF solution. After the deprotection of the eighth residue (Glu) was finished, one sixth of the resin was moved out to a 25 mL flitted glass tube, swollen with DMF and a 3-fold excess of BocBaa (157 mg, 0.15 mmol) was dissolved in 9 mL DMF/DCM (2:1). The Boc-Baa solution was first activated with PyBOP/HOBt/DIEA (1:1:1:3) for 2 minutes. The activated Boc Baa was mixed with the resin in the flitted glass tube, and shaken on an automated shaker for 1 day at room temperature. Then the resin was washed thoroughly with DMF and DCM to remove unreacted BocBaa. The final peptide was cleaved twice from the solid support using 10 mL TFA:TIPS:H₂O (95:2.5:2.5) for 4 h and 18 hrs. The crude fraction were washed with Et₂O and lyophilized to remove TFA. RP-HPLC purification was carried out on a Phenomenex Luna C5 column using an isocratic gradient of A: 0.1% TFA in water, and B: 0.1% TFA in isopropanol, 70% B, at 5.0 mL/min flow rate. The elution time was 42 min. After purification 8.8 mg (8.7%) were recovered. MALDI-MS: m/z, calculated 3399 [M⁺+2H], 3421 [M⁺+Na]. Found, 3400, 3421.

### Example 4

Baa-Lys-Lys-Lys-Lys-Lys-Lys-Lys-Lys-Lys-CO₂H (24). The couplings of first 8 residues after Baa of Fullerene peptide 1 was carried out on an automated APEX 396 Multiple Peptide Synthesizer (Advanced ChemTech) under nitrogen flow. Fmoc-Lys(Boc)-Wang resin (235 mg, 0.15 mM) was used as solid phase. Each coupling uses 4 fold amino acid excess, and HBTU, HOBt as activators and DIEA as base in a 1:1:1:3 ratio. Fmoc deprotection was performed using 25% piperidine in DMF solution. After the deprotection of the eighth residue (Glu) was finished, one sixth of the resin was moved out to a 25 mL fitted glass tube, swollen with DMF and a 3-fold excess of Boc-Baa (157 mg, 0.15 mmol) was dissolved in DMF/DCM (2:1, 9 mL). The Boc-Baa solution was first activated with PyBOP/HOBt/DIEA (1:1:1:3) for 2 minutes. The activated Boc-Baa was mixed with the resin in the fitted glass tube, and shaken on an automated shaker for 1 day at room temperature. Then the resin was washed thoroughly with DMF and DCM to remove unreacted BocBaa. The final peptide was cleaved twice from the solid support using 10 mL TFA:TIPS:H₂O (98:1:1) for 4 h and 18 hrs. The crude fraction were washed with Et₂O and lyophilized to remove TFA. RP-HPLC purification was carried out on a Phenomenex Luna C5 column using an isocratic gradient of A: 0.1% TFA in water, and B: 0.1% TFA in isopropanol, 70% B, at 5.0 mL/min flow rate. The elution time was 27 min. After purification 37.6 (35.7%) mg were recovered. MALDI-MS: m/z 2109 [M⁺+H]. Found 2109.

### Example 5

Baa-Lys(FITC)-Lys-Lys-Lys-Lys-Lys-Lys-Lys-Lys-CO₂H (25). The couplings of the natural amino acid sequence without Baa were carried out on an automated APEX 396 Multiple Peptide Synthesizer (Advanced ChemTech). Preloaded Fmoc-Lys(Boc)-Wang resin (469 mg, 0.30 mmol) was used as solid phase. Each coupling uses 4 fold amino acid excess, and HBTU, HOBt as activators and DIEA as base in a 1:1:1:3 ratio. Fmoc deprotection was performed using 25% piperidine in DMF solution. After the Lys₈ sequence (Lys-Lys-Lys-Lys-Lys-Lys-Lys-Lys) was completed, a Lys(Mtt) residue was coupled to the end. After the Fmoc deprotection of Lys(Mtt) was finished, one sixth of the resin was moved out to a 25 mL flitted glass tube, swollen with DMF. Then a 3-fold excess of Fmoc-Baa was (157 mg, 0.15 mM) dissolved in 9 mL DMF/DCM (2:1) in a glass vial. The Boc-Baa solution was first activated with PyBOP/HOBt/DIEA (1:1:1:3) for 2 minutes, then mixed with the resin in the fitted glass tube, and shaken on an automated shaker for 1 day at room temperature. Then the resin was washed thoroughly with DMF and DCM to remove any unreacted BocBaa. Prior to the addition of FITC fluorescence label, the resin was washed with DCM for complete removal of DMF. To achieve the maximum cleavage of Mtt protecting group, the resin was shrunk with MeOH twice. Then the resin was treated with 1% TFA and 5% TIPS in DCM 2 minutes for three times. The resin was washed again with DCM thoroughly, and swelled in DMF for 1 hour. Afterwards the resin was shaken with a solution of FITC (65 mg) in DMF (8 mL) and DIPEA (130 mL) overnight. At the end of the synthesis, the FITC labeled fullerene peptides was washed repeatedly with DMF, DCM and shrunk with MeOH. The resin was thoroughly dried over Driete in vacuo overnight. The cleavage of the peptide was achieved with TFA/TIPS/thiolanisole/H₂O (92.5:2.5:2.5:2.5) cocktail for 4 hr. After filtration, the peptide solution was concentrated by Rotary evaporation at room temperature and precipitated with cold diethyl ether. The crude was washed with Et₂O two more times and. After centrifugation of the final wash, it was frozen and lyophilized. RP-HPLC purification was carried out on a Phenomenex Luna C5 column using an isocratic gradient of A: 0.1% TFA in water, and B: 0.1% TFA in isopropanol, 70% B, at 5.0 mL/min flow rate. The elution time was 28 min. After purification 54.2 mg (43.4%) were recovered. MALDI-MS: m/z calculated 2496 [M⁺], 2107, [M⁺-FITC] found 2496, 2107.

### Example 6

Baa-Lys(FITC)-Pro-Lys-Lys-Lys-Arg-Lys-Val-OH (26). The couplings of normal amino acid sequence without Baa was carried out on an automated APEX 396 Multiple Peptide Synthesizer (Advanced ChemTech). Preloaded Fmoc-Val-Wang resin (491 mg, 0.30 mmol) was used as solid phase. Each coupling uses 4 fold amino acid excess, and HBTU, HOBt as activators and DIEA as base in a 1:1:1:3 ratio. Fmoc deprotection was performed using 25% piperidine in DMF solution. After the NLS sequence (Pro-Lys-Lys-Lys-Arg-Lys-Val) was completed, a Lys(Mtt) residue was coupled to the end. After the Fmoc deprotection of Lys(Mtt) was finished, one sixth of the resin was moved out to a 25 mL fritted glass tube, swollen with DMF. Then a 3-fold excess of Fmoc-Baa was (157 mg, 0.15 mM) dissolved in DMF/DCM (9 mL, 2:1) in a glass vial. The Boc Baa solution was first activated with PyBOP/HOBt/DIEA (1:1:1:3) for 2 minutes, then mixed with the resin in the flitted glass tube, and shaken on an automated shaker for 1 day at room temperature. Then the resin was washed thoroughly with DMF and DCM to remove any unreacted BocBaa. Prior to the addition of FITC fluorescence label, the resin was washed with DCM for complete removal of DMF. To achieve the maximum cleavage of Mtt protecting group, the resin was shrunk with MeOH twice. Then the resin was treated with 1% TFA and 5% TIPS in DCM 2 minutes for three times. The resin was washed again with DCM thoroughly, and swelled in DMF for 1 hour. Afterwards the resin was shaken with a solution of FITC (65 mg) in DMF (8 mL) and DIEA (130 mL) overnight. At the end of the synthesis, the FITC labeled fullerene peptides was washed repeatedly with DMF, DCM and shrunk with MeOH. The resin was thoroughly dried over Driete in vacuo overnight. The Cleavage of the peptide was achieved with TFA/TIPS/H₂O (95:2.5:2.5) cocktail for 4 hr. After filtration, the peptide solution was concentrated by Rotary evaporation at room temperature and precipitated with cold Et₂O. The crude was washed with diethyl ether two more times and. After centrifugation of the final wash, it was frozen and lyophilized. RP-HPLC purification was carried out on a Phenomenex Luna C5 column using an isocratic gradient of A: 0.1% TFA in water, and B: 0.1% TFA in isopropanol, 70% B, at 5.0 mL/min flow rate. The elution time was 37 min. After purification 59.1 mg (50.6%) were recovered. MALDI-MS: m/z calculated 2337 [M+₊H], 1948 [M⁺+H-FITC]. Found 2337, 1948.

### Example 7

Baa-Glu-Glu-Glu-Glu-Gly-Gly-Gly-Ser-CO₂H (27). The couplings of standard amino acids were carried out on an automated APEX 396 Multiple Peptide Synthesizer (Advanced ChemTech) under nitrogen flow. Fmoc-Serine(tBu)-Wang resin (469 mg, 0.3 mM) was used as solid phase. Each coupling uses 4 fold amino acid excess, and HBTU, HOBt as activators and DIEA as base in a 1:1:1:3 ratio. Fmoc deprotection was performed using 25% piperidine in DMF solution. After the deprotection of the eighth residue (Glu) was finished, one sixth of the resin was moved out to a 25 mL fritted glass tube, swollen with DMF and a 3-fold excess of Boc-Baa (157 mg, 0.15 mmol) was dissolved in 9 mL DMF/DCM (2:1). The Boc-Baa solution was first activated with PyBOP/HOBt/DIEA (1:1:1:3) for 2 minutes. The activated Boc-Baa was mixed with the resin in the flitted glass tube, and shaken on an automated shaker for 1 day at room temperature. Then the resin was washed thoroughly with DMF and DCM to remove unreacted BocBaa. The final peptide was cleaved twice from the solid support using 10 mL TFA:TIPS:H₂O (98:1:1) for 4 h and 18 hrs. The crude fraction were washed with dietheyl ether and lyophilized to remove TFA. RP-HPLC purification was carried out on a Phenomenex Luna C5 column using an isocratic gradient of A: 0.1% TFA in water, and B: 0.1% TFA in isopropanol, 70% B, at 5.0 mL/min flow rate. The elution time was 43 min. After purification 21.8 mg (24.9%) were recovered. MALDI-MS: m/z 1752 [M++Na]. Found 1752.

### Example 8

Baa-Lys(FITC)-Glu-Glu-Glu-Glu-Gly-Gly-Gly-Ser-CO₂H (28). Glu-Glu-Glu-Glu-Gly-Gly-Gly-Ser-Wang resin (Ca. 0.05 mM) prepared from 27 was moved out to a 25 mL fritted glass tube, swollen with DMF and a 3-fold excess of Boc-Baa (157 mg, 0.15 mmol) was dissolved in 9 mL DMF/DCM (2:1). The Boc-Baa solution was first activated with PyBOP/HOBt/DIEA (1:1:1:3) for 2 minutes. The activated Boc-Baa was mixed with the resin in the fritted glass tube, and shaken on an automated shaker for 1 day at room temperature. Then the resin was washed thoroughly with DMF and DCM to remove unreacted BocBaa. Prior to the addition of FITC fluorescence label, the resin was washed with DCM for complete removal of DMF. To achieve the maximum cleavage of Mtt protecting group, the resin was shrunk with methanol twice. Then the resin was treated with 1% TFA and 5% TIPS in DCM for 2 minutes for three times. The resin was washed again with DCM thoroughly, and swelled in DMF for 1 hour. Afterwards the resin was shaken with a solution of FITC (65 mg) in DMF (8 mL) and DIPEA (130 mL) overnight. At the end of the synthesis, the FITC labeled fullerene peptides was washed repeatedly with DMF, DCM and shrunk with MeOH. The resin was thoroughly dried over Driete in vacuo overnight. The cleavage of the peptide was achieved with TFA/TIPS/H₂O (95:2.5:2.5) cocktail for 4 hr. After filtration, the peptide solution was concentrated by Rotary evaporation at room temperature and precipitated with cold Et₂O. The crude was washed with diethyl ether two more times and. After centrifugation of the final wash, it was frozen and lyophilized. RP-HPLC purification was carried out on a Phenomenex Luna C5 column using an isocratic gradient of A: 0.1% TFA in water, and B: 0.1% TFA in isopropanol, 70% B, at 5.0 mL·min⁻¹ flow rate. The elution time was 33 min. After purification 8.4 mg (7.4%) were recovered. MALDI-MS: m/z calculated 2270 [M++Na]. Found 2271.

### Example 9

Baa-Arg-Gln-Ile-Lys-Ile-Trp-Phe-Gln-Asn-Arg-Arg-Met-Lys-Trp-Lys-Lys-OH (29). The couplings of penetratin sequence without Baa was carried out on an automated APEX 396 Multiple Peptide Synthesizer (Advanced ChemTech) under nitrogen flow. Fmoc-Lys-Wang resin (448 mg, 0.3 mmol) was used as solid phase. Each coupling uses 4 fold amino acid excess, and HBTU, HOBt as activators and DIEA as base in a 1:1:1:3 ratio. Fmoc deprotection was performed using 25% piperidine in DMF solution. After the deprotection of the 15th residue (Arg) was finished, one sixth of the resin was moved out to a 25 mL fritted glass tube, swollen with DMF and a 3-fold excess of BocBaa (157 mg, 0.15 mmol) was dissolved in 9 mL DMF/DCM (2:1). The Boc-Baa solution was first activated with PyBOP/HOBt/DIEA (1:1:1:3) for 2 minutes. The activated Boc-Baa was mixed with the resin in the fritted glass tube, and shaken on an automated shaker for 1 day at room temperature. Then the resin was washed thoroughly with DMF and DCM to remove unreacted BocBaa. The final peptide was cleaved twice from the solid support using 10 mL TFA:TIPS:H₂O (98:1:1) for 4 hrs. The crude fraction were precipitated and washed with Et₂O and lyophilized to remove TFA. RP-HPLC purification was carried out on a Phenomenex Luna C5 column using an isocratic gradient of A: 0.1% TFA in water, and B: 0.1% TFA in isopropanol, 70% B, at 5.0 mL/min flow rate. The elution time was 33 min. After purification 58.7 mg (36.9%) were recovered. MALDI-MS: m/z calculated 3184 [M⁺+H]. Found 3184.

### Example 10

Baa-Glu-Glu-Glu-Glu-Gly-Gly-Gly-Gly-Ser-Cys-OH (210). The couplings of E₄G₃SC sequence without Baa was carried out on an automated APEX 396 Multiple Peptide Synthesizer (Advanced ChemTech) under nitrogen flow. Fmoc-Cys-Wang resin (510 mg, 0.3 mmol) was used as solid phase. Each coupling uses 4 fold amino acid excess, and HBTU, HOBt as activators and DIEA as base in a 1:1:1:3 ratio. Fmoc deprotection was performed using 25% piperidine in DMF solution. After the deprotection of the 8th residue (Glu) was finished, one sixth of the resin was moved out to a 25 mL fritted glass tube, swollen with DMF and a 3-fold excess of BocBaa (157 mg, 0.15 mM) was dissolved in 9 mL DMF/DCM (2:1). The Boc-Baa solution was first activated with PyBOP/HOBt/DIEA (1:1:1:3) for 2 minutes. The activated Boc-Baa was mixed with the resin in the flitted glass tube, and shaken on an automated shaker for 1 day at room temperature. Then the resin was washed thoroughly with DMF and DCM to remove unreacted BocBaa. The final peptide was cleaved twice from the solid support using 10 mL TFA:TIPS:H₂O (98:1:1) for 4 hrs. The crude fraction were precipitated and washed with dietheyl ether and lyophilized to remove TFA. RP-HPLC purification was carried out on a Phenomenex Luna C5 column using an isocratic gradient of A: 0.1% TFA in water, and B: 0.1% TFA in isopropanol, 70% B, at 5.0 mL/min flow rate. The elution time was 37 min. After purification 8.0 mg (8.5%) were recovered. MALDI-MS: m/z 1889 [M⁺], 1912 [M⁺+Na]. Found 1889, 1911

### Example 11

Baa-Lys(FITC)-Pro-Lys-Lys-Lys-Arg-Lys-Val-Ser-Cys-OH (211). The couplings of normal amino acid sequence without Baa was carried out on an automated APEX 396 Multiple Peptide Synthesizer (Advanced ChemTech). Preloaded Fmoc-Cys Wang resin (510 mg, 0.30 mmol) was used as solid phase. Each coupling uses 4 fold amino acid excess, and HBTU, HOBt as activators and DMA as base in a 1:1:1:3 ratio. Fmoc deprotection was performed using 25% piperidine in DMF solution. After the NLS sequence (Pro-Lys-Lys-Lys-Arg-Lys-Val) was completed, a Lys(Mtt) residue was coupled to the end. After the Fmoc deprotection of Lys(Mtt) was finished, one sixth of the resin was moved out to a 25 mL flitted glass tube, swollen with DMF. Then a 3-fold excess of Boc-Baa was (157 mg, 0.15 mM) dissolved in 9 mL DMF/DCM (2:1) in a glass vial. The Boc Baa solution was first activated with PyBOP/HOBt/DIEA (1:1:1:3) for 2 minutes, then mixed with the resin in the flitted glass tube, and shaken on an automated shaker for 1 day at room temperature. Then the resin was washed thoroughly with DMF and DCM to remove any unreacted BocBaa. Prior to the addition of FITC fluorescence label, the resin was washed with DCM for complete removal of DMF. To achieve the maximum cleavage of Mtt protecting group, the resin was shrunk with MeOH twice. Then the resin was treated with 1% TFA and 5% TIPS in DCM for 2 minutes three times. The resin was washed again with DCM thoroughly, and swelled in DMF for 1 hour. Afterwards the resin was shaken with a solution of FITC (65 mg) in DMF (8 mL) and DIPEA (130 mL) overnight. At the end of the synthesis, the FITC labeled fullerene peptides was washed repeatedly with DMF, DCM and shrunk with MeOH. The resin was thoroughly dried over Driete in vacuo overnight. The cleavage of the peptide was achieved with TFA/TIPS/thiolanisole/H₂O (92.5:2.5:2.5:2.5) cocktail for 4 hr. After filtration, the peptide solution was concentrated by rotary evaporation at room temperature and precipitated with cold diethyl ether. The crude was washed with Et₂O two more times and. After centrifugation of the final wash, it was frozen and lyophilized. RP-HPLC purification was carried out on a Phenomenex Luna C5 column using an isocratic gradient of A: 0.1% TFA in water, and B: 0.1% TFA in isopropanol, 70% B, at 5.0 mL/min flow rate. The elution time was 35 min. After purification 34.5 mg (27.3%) were recovered. MALDI-MS: m/z calculated 2528 [M⁺+H]. Found 2528.

### Example 12

Baa-(Lys)₁₀Lys(FITC)-Pro-Lys-Lys-Lys-Arg-Lys-Val-Ser-Cys-OH (212). Ca. 0.05 mM of Lys(Mtt)-Pro-Lys-Lys-Lys-Arg-Lys-Val-Cys-Wang resin prepared from 211 was retained in the peptide synthesizer. Ten Lys(Boc) residues were coupled thereafter. After the Fmoc deprotection of last Lys(Boc) was finished, the resin was moved out to a 25 mL flitted glass tube, swollen with DMF. Then a 3-fold excess of Boc-Baa was (0.15 mM, 157 mg) dissolved in 9 mL DMF/DCM (2:1) in a glass vial. The Boc-Baa solution was first activated with PyBOP/HOBt/DIEA (1:1:1:3) for 2 minutes, then mixed with the resin in the fritted glass tube, and shaken on an automated shaker for 1 day at room temperature. Then the resin was washed thoroughly with DMF and DCM to remove any unreacted Boc-Baa. The FITC tag was linked to the peptide chain following the same procedure as 211. At the end of the synthesis, the FITC labeled fullerene peptides was washed repeatedly with DMF, DCM and shrunk with MeOH. The resin was thoroughly dried over Driete in vacuo overnight. The cleavage of the peptide was achieved with TFA/TIPS/thiolanisole/H₂O (92.5:2.5:2.5:2.5) cocktail for 4 hr. After filtration, the peptide solution was concentrated by Rotary evaporation at room temperature and precipitated with cold diethyl ether. The crude was washed with Et₂O two more times. After centrifugation of the final wash, it was frozen and lyophilized. RP-HPLC purification was carried out on a Phenomenex Luna C5 column using an isocratic gradient of A: 0.1% TFA in water, and B: 0.1% TFA in isopropanol, 70% B, at 5.0 mL·min⁻¹ flow rate. The elution time was 25 min. After purification 98.3 mg (51.6%) were recovered. MALDI-MS: m/z 3418 [M⁺-FITC+H], 3809 [M⁺+H] found 3418, 3807.

### Example 13

Peptide stability. In order to test the stability of the fullerene peptide linkage, samples of Baa-Lys(FITC)-Lys₈-OH and H-Baa-Lys(FITC)-NLS were exposed to bovine plasma for up to 3 days and analyzed by HPLC. No degradation of the sample was observed suggesting that both the fullerene amine linkage and the Baa-peptide linkages are stable under conditions relevant to the present study.

### Example 14

Peptide aggregation studies. It has been observed that the parent amino acid, Baa, aggregates in aqueous solution as a consequence of the presence of both hydrophobic and hydrophilic groups within the same molecule. It is reasonable to suppose that the peptides that are soluble in water would also form aggregates. The state of aggregation may have an impact on cell penetration and thus, aggregation was studied in detail.

With reference back to Table 1, in order to understand the intracellular transport of the FITC functionalized Baa-peptides, 25 and Baa-Lys(FITC)-NLS (26), their particle size in solution was determined by dynamic light scattering (DLS). Three series of solutions of 24, 25 and 26 in the range of 0.125-2.0 mg/mL was prepared by weighing method. PBS buffer prepared from HPLC grade water was used as solvent and was filtered through a 0.10 mm cup filter (Millipore, Express).

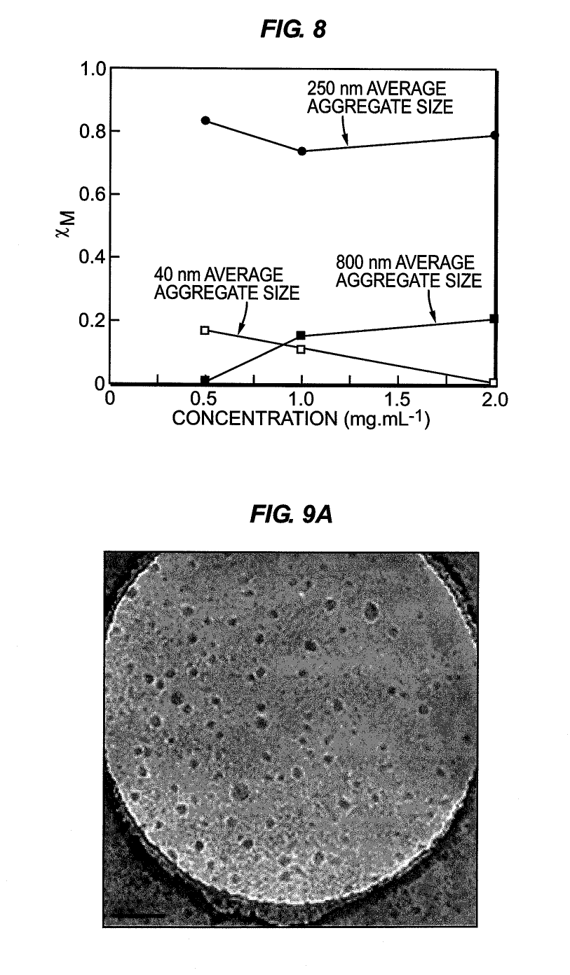

The solution aggregation of 25 was compared with its non-FITC containing analog (24) to determine the effects of the FITC. Peptides 24, 25, and 26 all show aggregation in aqueous solution across the concentration ranges measured. The polylysine peptide 24 exhibits a single broad aggregate distribution (50-350 nm) with an average size of ca. 200 nm. The size of the aggregate is independent of concentration (0.25-2.0 mg·mL⁻¹), although the distribution narrows with increased concentration. In contrast, FITC-labeled poly-lysine peptide 25 shows two distinct aggregate sizes at concentrations between 0.125 and 1.0 mg/mL. The most major component (ca. 60%) is comparable in size (ca. 180 nm) to that seen for 24. The minor component is a smaller aggregate (ca. 10 nm) with a relatively narrow distribution (5-20 nm). At high concentrations (2.0 mg/mL) a third larger aggregate (ca. 1500 nm) is observed at the expense of the both of the other aggregate sizes, as shown in FIG. 7. FIG. 7 is a plot of the fraction of aggregates for Baa-Lys(FITC)-Lys₈-OH (25) as a function of solution concentration. A similar trend is observed for Baa-Lys(FITC)-NLS (26) (FIG. 8). At 0.5 mg·mL⁻¹ there appears to be two distinct types of aggregate; the major species (ca. 80%) is ca. 250 nm while the minor content is again a smaller aggregate (ca. 40 nm). As may be seen from FIG. 8, above 1.0 mg·mL⁻¹ a third distinct aggregate is observed of ca. 800 nm. The average size and distribution of each type of aggregate does not change significantly with concentration.

It would appear that the major solution species for peptides 24, 25, and 26 (>60%) are aggregates of between 180 and 250 nm in size. The size of these aggregates is not dependant on the concentration, however, the presence of the FITC substituent results in two minor types of aggregate to be observed. Both of these are presumably do to the potential packing interactions of the FITC residue, either with itself or the fullerenes.

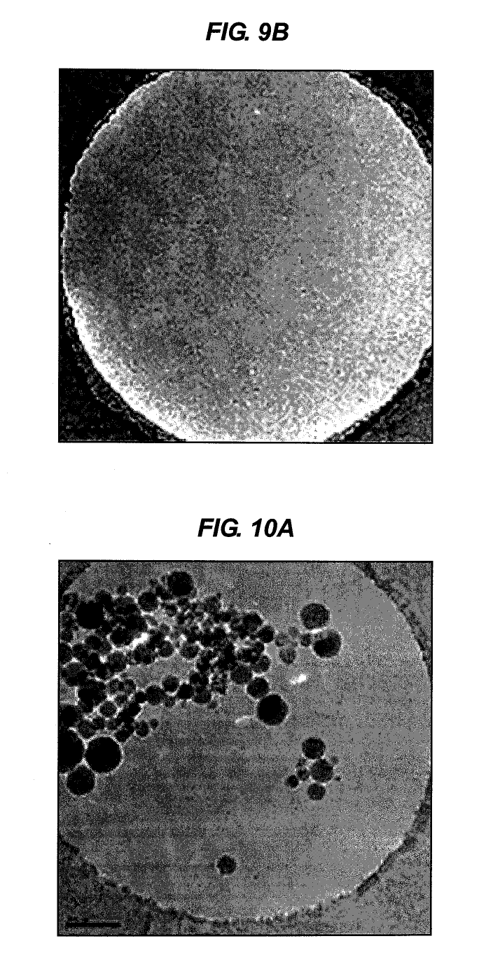

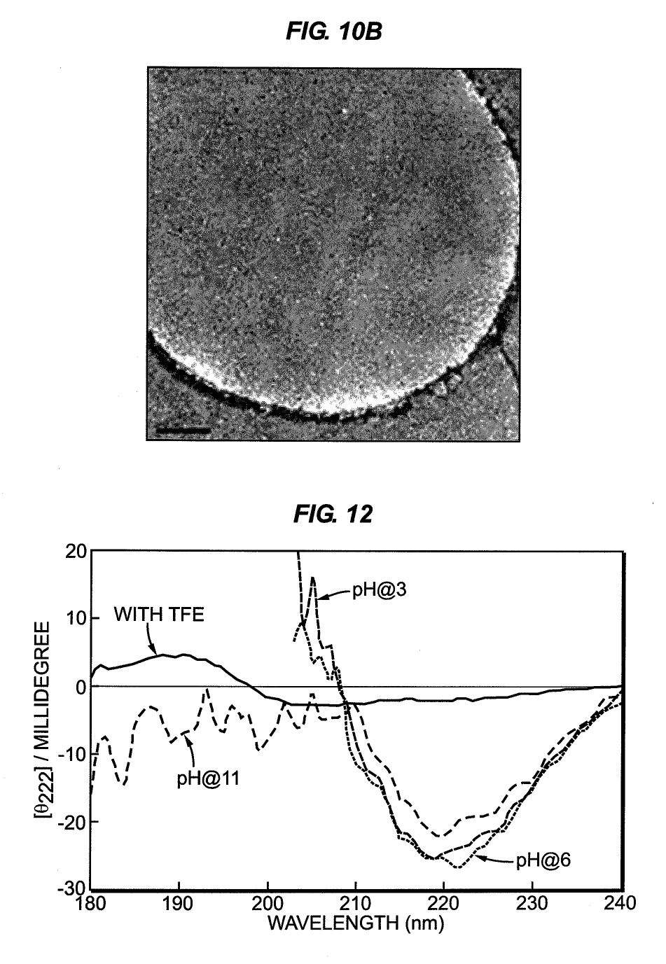

To further examine the actual aggregate size and morphology, cryo-TEM experiments were performed for both peptides 25 and 26. The images were taken in the concentration of 1.0 mg/mL for both peptides. Samples for cryo-TEM studies were prepared by dipping a copper grid coated with amorphous carbon-holey film into the sample solution. The TEM images were mainly taken in the hole region of the TEM grid to minimize the artificial effect from the samples or ice. The result showed that fullerene peptides exhibited strong aggregation behavior in aqueous solution, a similar phenomenon demonstrated by other water-soluble fullerene derivatives. Both peptides forms spherical and ellipsoidal clusters, with an average aggregate sizes of 40-80 nm for 25 and 50-150 nm for 26, which are generally smaller than the diameters observed by DLS. Consistent with the DLS study, Baa-Lys(FITC)-Lys₈-OH (25) are more uniform in size than Baa-Lys(FITC)-NLS (26). FIGS. 9a and 9b show the vitreous ice cryo-TEM micrograph of the large aggregates (9a) and small aggregates (9b) formed by Baa-Lys(FITC)-Lys₈-OH in PBS buffer (1 mg/mL at pH=7). There are two dominant groups in size for 25; one type has the diameter of 40-80 nm with predominant population at size of ca. 50 nm, and the other population has a diameter less than 20 nm, which may correspondent to the size of fullerene peptides itself. In contrast, the size distribution of 26 large aggregates is much polydisperse, ranging from 50-150 nm with no obvious dominant population for large aggregates, while smaller aggregates has similar size as Baa-Lys(FITC)-Lys₈-OH. FIGS. 10a and 10b show the vitreous ice cryo-TEM micrograph of the large aggregates (10a) and small aggregates (10b) formed by Baa-Lys(FITC)-NLS in PBS buffer (1 mg/mL at pH=7). The aggregate sizes from the two peptides seem to be consistent with the hydrophilicity of peptide chain as more hydrophilic sequence corresponds to smaller size (25), and vice versa (26).

The presence of the FITC ligand has a significant impact on the aggregation and self-assembly of the Baa-containing peptides. It should be noted that these results might have implications for the cellular uptake of the peptides as SDS can be viewed as the simplest form of cell membrane.

### Example 15

Control of peptide secondary structure by fullerene amino acids. In an effort to understand the effect of the C₆₀ substituents on the secondary structure of a peptide we have investigated two sequences and the position within a particular sequence. The well characterized model heptad peptides Glu-Ile-Ala-Gln-Leu-Glu-Tyr-Glu-Ile-Ser-Gln-Leu-Glu-Gln-NH₂ (2HP, 22 in Table 1) Glu-Ile-Ala-Gln-Leu-Glu-Tyr-Glu-Ile-Ser-Gln-Leu-Glu-Gln-Gln-Glu-Ile-Gln-Ala-Leu-Glu-Ser-NH₂ (3HP, 23 in Table 1) were chosen because they are known to form fibers at pH below 7 concomitant with β-sheet formation. To investigate the effects of the presence and C₆₀ on the conformation of the peptide the model peptide 2HP was prepared in which either tyrosine was replaced by Baa (21 in Table 1) or Baa was added to the C-terminus (22 in Table 1). The C-terminus derivative 23 was also prepared for 3HP. The results are shown in Table 2 below.

TABLE 2

| | Secondary structure with | |
|---|---|---|
| Peptide | decreasing pH | Appearance |
| 2HP | random coil → α-helix → β-sheet | fibers |
| 21 | β-sheet | fibers^{a} |
| 22 | random coil → α-helix | aggregates |
| 3HP | random coil → α-helix | fibers |
| 23 | α-helix coiled | fibers |

The parent 2HP adopts a random coil configuration above pH 7. As the pH is lowered it exhibits a α-helix structure as a transition to the formation of β-sheets below pH 7. TEM studies show that the β-sheet form exhibits a fiber like structure. The addition of the Baa, irrespective of the position, has a dramatic effect on the relative stability of the peptide secondary structure as compared to 2HP; however, the position of substitution alters the mode of the effect.

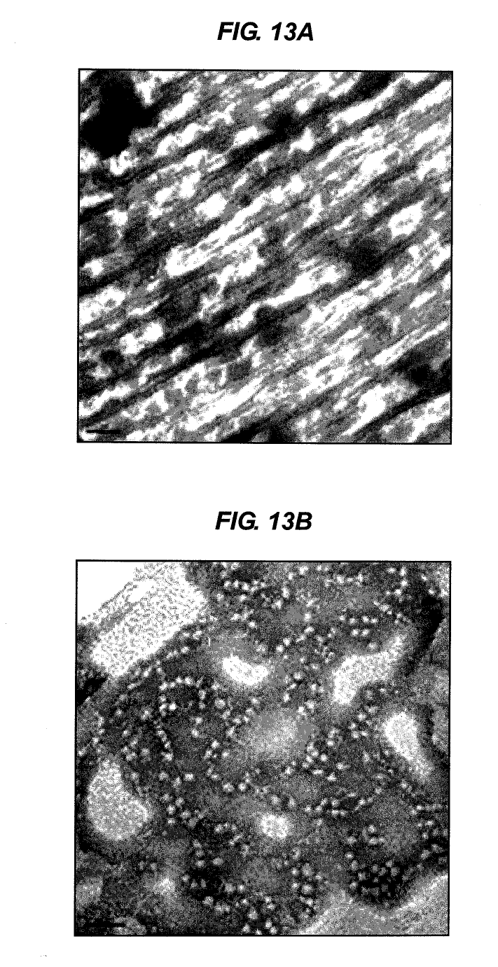

Positioning the Baa in the center of the 2HP sequence by replacement of tyrosine (i.e., 21) resulted a circular dichroism (CD) consistent with the formation of a 13-sheet structure across the pH range 3-11 (FIG. 12). Thus, the presence of the C₆₀ has a clear stabilization effect on the stability of the β-sheet conformation. Note that the CD does not exclude the presence of other conformations, but indicates the β-sheet is the major conformation. As with the parent 2HP, peptide 21 forms fibrous structures. However, there is a minor component of a non-fibrous structure observed within the sample. FIGS. 13a and 13b show TEM images of the fullero-peptide 21 precipitated onto a lacy carbon grid at pH 4 with the fibrous component (FIG. 13a) and the minor micellar component (FIG. 13b). The non-fibrous micelles appear to be 8-10 nm in diameter. The conversion from β-sheet to α-helix may be induced by the addition of CF₃CH₂OH (see FIG. 12, TFE).

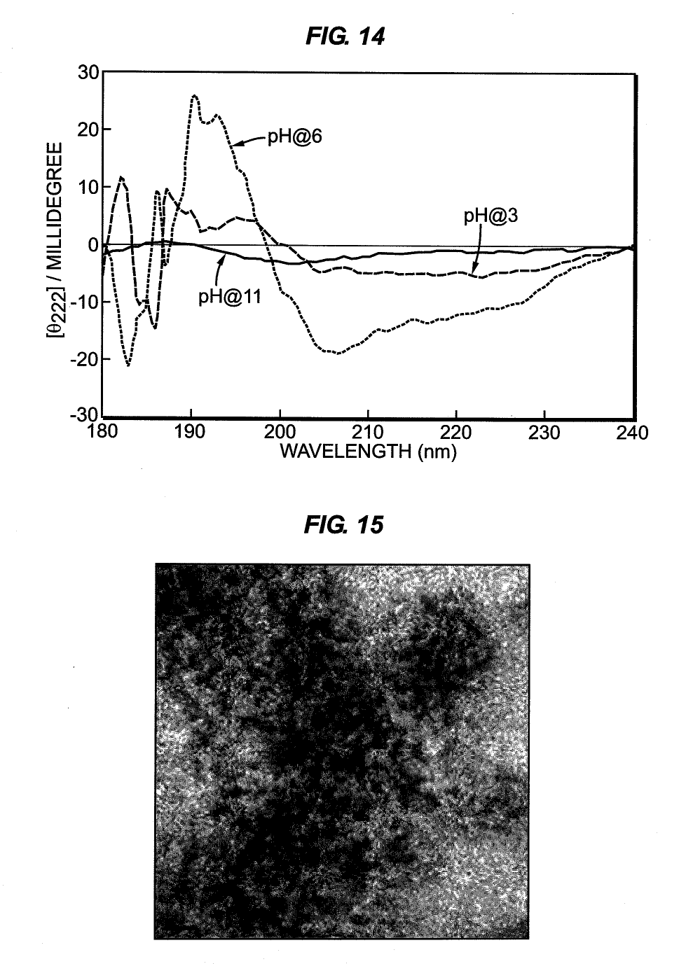

In contrast to the results for substitution in the center of the peptide, addition of Baa to the C-terminus of 2HP results in a peptide (22) with a CD spectrum that indicates the formation of a weak random coil under basic conditions (see FIG. 14), i.e., in a similar manner to the parent 2HP. Upon reduction of the solution pH the structure of 22 transforms to an α-helix, however, unlike 2HP, peptide 22 does not subsequently convert to a β-sheet conformation. Instead, slow precipitation of an aggregate occurs at pH 6 with more rapid precipitation of the same material occurring at lower pH (FIG. 14). From the TEM images this precipitate appears to comprise of a network of particles of approximately 20 nm in diameter as shown in FIG. 15.

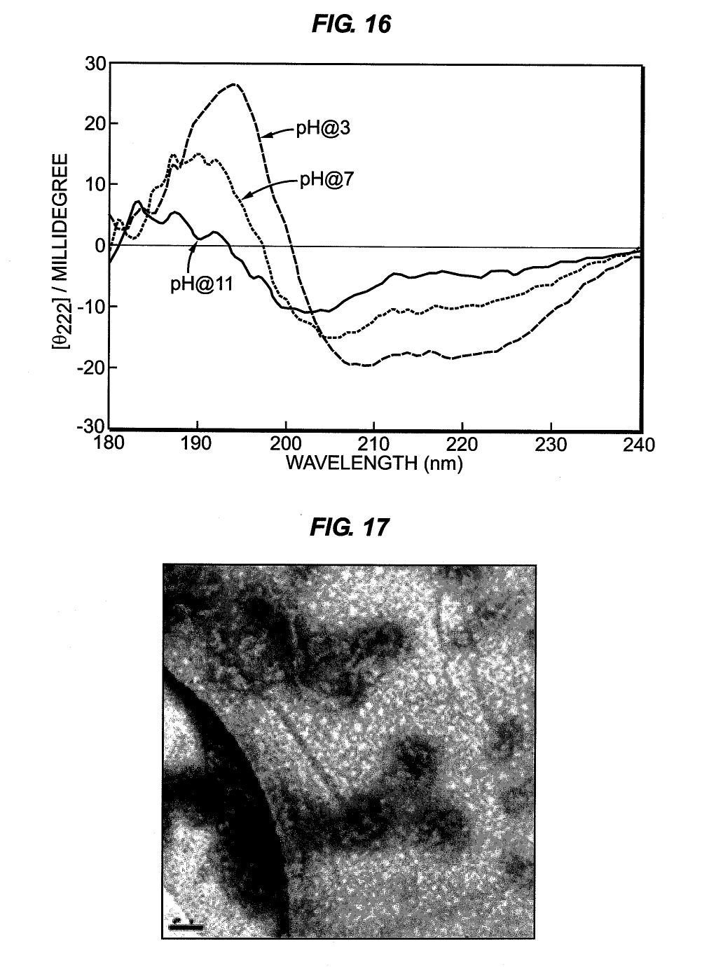

The solution conformation of 3HP at high pH is that of a random coil, however, with decreasing pH an α-helix is formed that results in the formation of a short fiber-like structure: upon aging longer fibers are formed. In contrast the C-termini Baa-functionalized 3HP (23) shows the formation of a weak α-helix structure even at high pH as shown in FIG. 16. The TEM images of FIG. 17 show the formation of coiled (“curly”) fiber.

The presence of the strongly hydrophobic C₆₀ substituent has a dramatic influence on the structural stability of various secondary structures. Based upon these initial examples it is possible to make a broad generalization as to the effect of the fullerene. End substitution or addition results in the promotion and stabilization of the α-helix structure. In contrast, substitution in the middle of the peptide results in promotion and stabilization of a β-sheet structure.

### Example 16

Antioxidant properties of fullerne-peptides. Given that the parent fullero-amino acid, Baa, has been shown to exhibit strong antioxidant properties, we are interested if a Baa containing peptide retains the antioxidant properties.

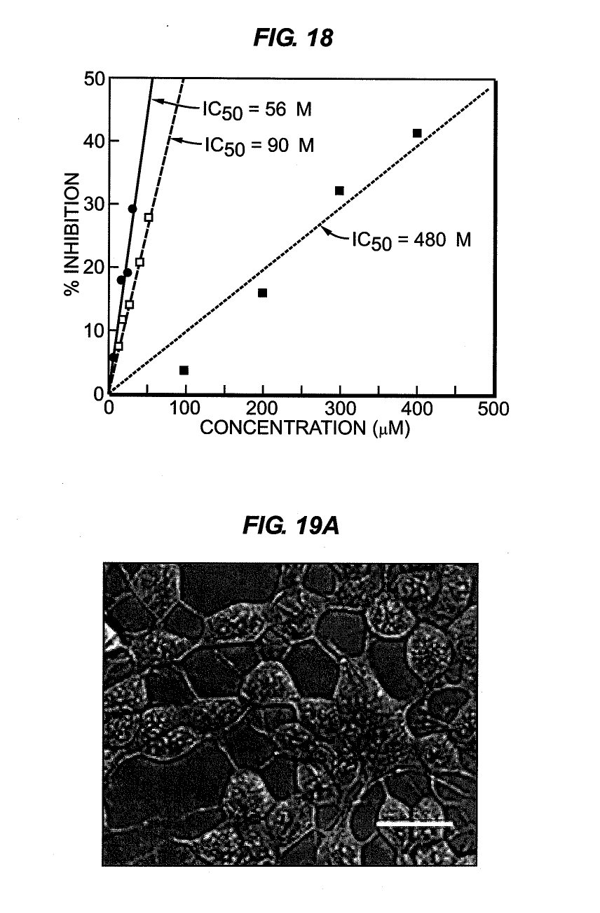

The IC₅₀ of Baa-Glu-Glu-Glu-Glu-Gly-Gly-Gly-Ser-OH was determined by Csixty Inc. to be 89 μM through an established method. A plot of inhibition as a function of concentration for peptide 27 is shown in FIG. 18 with a comparison of Baa and Trolox. Based upon this assay it is apparent that while the anionic peptide Baa-Glu-Glu-Glu-Glu-Gly-Gly-Gly-Ser-OH has ca. five fold the activity of Trolox (IC₅₀=480 μM), its molar activity is not as good as Baa itself (IC₅₀=55 μM). It is unclear at the present time the reason for the slight reduction in activity between Baa and Baa-Glu-Glu-Glu-Glu-Gly-Gly-Gly-Ser-OH. Given that the peptide's aggregation in aqueous solution presumably results in the “Glu-Glu-Glu-Glu-Gly-Gly-Gly-Ser-OH” being exposed to the solution, the sequence could act as a buffer limiting electron transfer processes.

### Example 17

Cellular uptake. An immortalized human embryonic kidney epithelial cancer cell line was used in initial cellular uptake studies. HEK-293a cells were cultured in RMPI 1640 medium supplemented with 10% fetal bovine serum (FBS) in 5% CO₂ and 37° C. humidified incubator. The medium was supplemented with penicillin (100 U·mL⁻¹), streptomycin (100 μg/mL), and glutamine (2 mM). For experiments and microscopy, cells were seeded at 1×10⁴ cells per well on 6-well plates and grown for two days in RPMI supplemented with 10%. Cells were incubated with each peptide (40 μM) for 24 hrs at 37° C. After treatment, the cells were washed with RPMI supplemented with 10% FBS and phosphate buffered saline (PBS, 10 mM). Fluorescence was observed by a fluorescent microscope equipped with FITC and red filters.

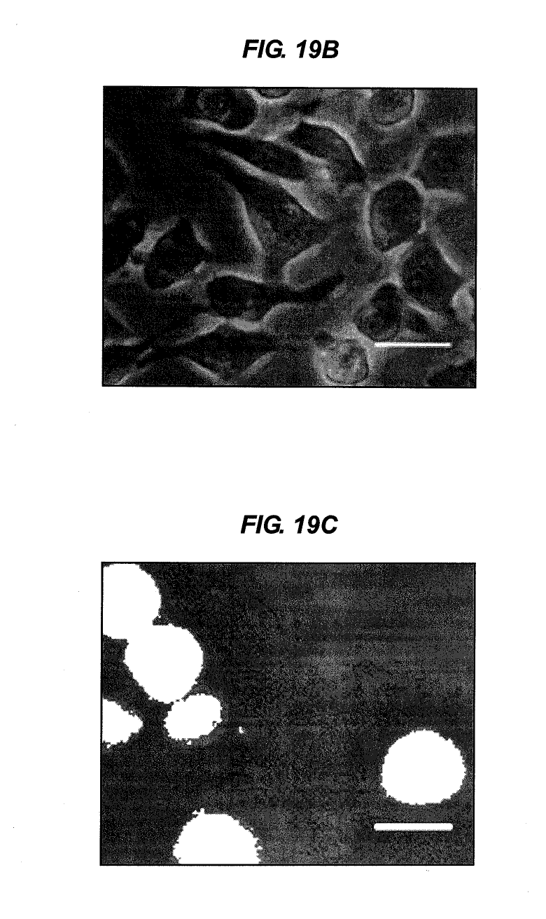

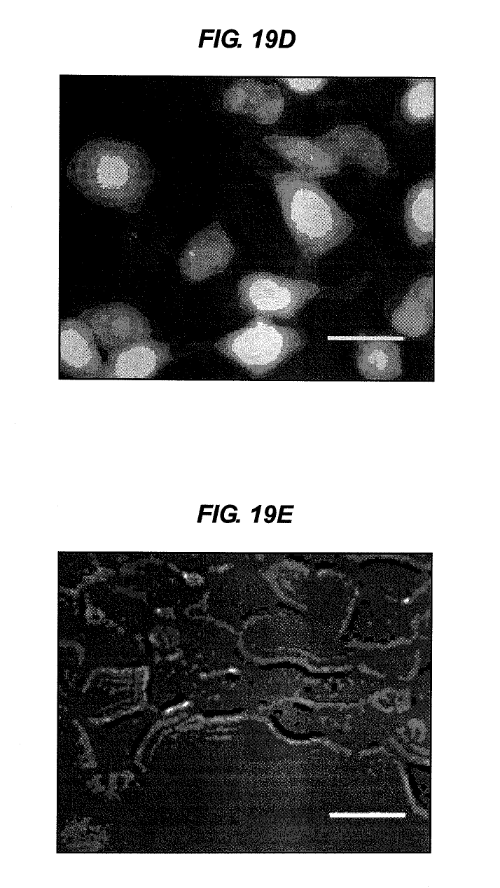

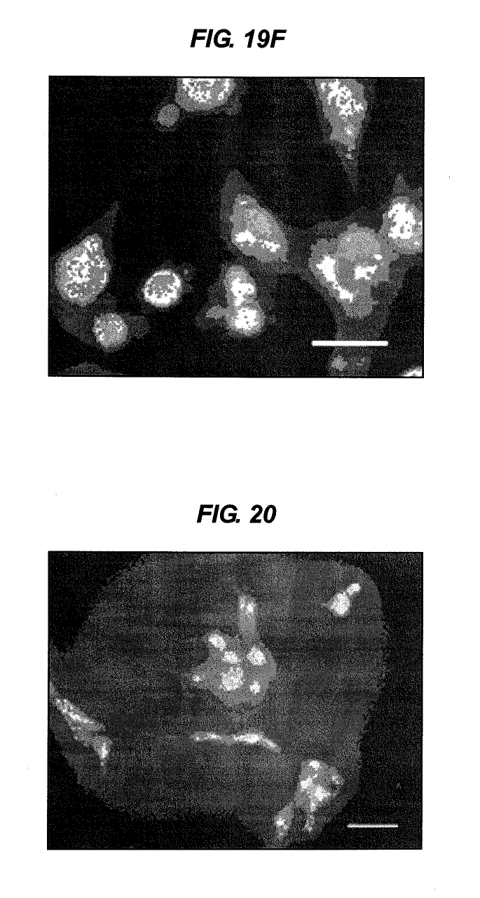

Optical microscope images of HEK-293a cells incubated with H-Lys(FITC)-Lys₈-OH (FIG. 19a) and H-Baa-Lys(FITC)-Lys₈-OH (FIG. 19b) are indistinguishable. In contrast, the fluorescence images show a dramatic difference. The oligolysine peptide, H-Lys(FITC)-Lys₈-OH (FIG. 19c), shows no uptake in the cells by fluorescence while H-Baa-Lys(FITC)-Lys₈-OH (FIG. 19d) shows strong green fluorescence within the cytoplasm. Thus, while the cationic peptide H-Lys(FITC)-Lys₈-OH shows no ability to cross over the cell membrane, the addition of the Baa amino acid residue facilitates the intracellular localization of the peptide (Table 3).

TABLE 3

| | Lys(FITC)- | Baa-Lys(FITC)- | Lys(FITC)- | Baa-Lys(FITC)- |
|---|---|---|---|---|
| Peptide | NLS | NLS | (Lys)₈-OH | (Lys)₈-OH |
| Active | No | Yes | No | Yes |
| Cellular | | | | |
| Uptake | | | | |

It has been reported that Lys(FITC)-NLS shows no uptake into cells in the absence of a conjugate. F. Noor, A. Wustholz, R. Kinscherf, N. Metzler-Nolte, Angew. Chem. Int. Ed. 44, 2429-2432 (2005). Our phenylalanine derivative, H-Phe-Lys(FITC)-NLS, shows a similar lack of uptake into the HEK-293a cells (FIG. 19e). In direct contrast, H-Baa-Lys(FITC)-NLS shows a localized intense fluorescence in the center of the cells (FIG. 19f). Treatment with DAPI nuclei staining dye showed that while there is a correlation between the location of the H-Baa-Lys(FITC)-NLS and the nuclei, the peptide is not located exclusively within the nuclei. It would appear therefore that the H-Baa-Lys(FITC)-NLS is located in the nucleus region of the cell, but transport across the nuclear membrane is not extensive under the present conditions. Given the aggregation of the fullerene peptides it is possible that the transport across the nuclear membrane is inhibited by the size of the aggregates.

The uptake into the cells for both H-Baa-Lys(FITC)-NLS and H-Baa-Lys(FITC)-Lys₈-OH was found to be temperature dependent. Cell uptake studies performed at 4° C. showed no cellular uptake activity for either fullerene peptide. These results suggest that the cellular uptake activity of the fullerene peptides is an energy dependent process, which is a typically characteristic of an endocytosis process. It has been previously suggested that the endocytic translocation of the cell penetrating peptides (CPPs) is triggered by the electrostatic interaction of their net positive charge with the negatively charged phospholipid membrane. D. Derossi, S. Calvet, A. Trembleau, A. Brunissen, G. Chassaing, A. Prochiantz, J. Biol. Chem. 271, 18188-18193 (1996). If a similar process is occurring for the fullerene peptides then the use of a negatively charged sequence should reduce or preclude the cellular uptake. Cellular studies show that the uptake intensity for Baa-Lys(FITC)Glu₄Gly₃Ser was greatly reduced in comparison with the cationic fullerene peptides of the same concentration. These observations are consistent with endocytic internalization as the underlying translocation mechanism.

In order to show the generality of our results we have also studied the uptake of H-Baa-Lys(FITC)-NLS into neuroblastoma cell line (IMR 32). Neuroblastoma is the most common extracranial solid tumor in children and is responsible for 8-10% of pediatric tumors and 15% of pediatric cancer deaths. Neuroblastoma cells are known for their difficulty in transfection through the cell membrane. H-Baa-Lys(FITC)-NLS was incubated with IMR 32 cells for 24 hrs at 37° C. The cells were then washed with PBS buffer and treated with DAPI nuclei staining dye prior to observation with a fluorescence microscope. FIG. 20 shows intense point fluorescence in cytoplasm, and homogeneous intense fluorescence around nuclei closely associated with the blue of the DAPI nuclei staining dye. As was observed for the HEK-293 cells there is a correlation between the localization of H-Baa-Lys(FITC)-NLS and the nucleus, but it is obvious that green fluorescence did not internalize into the nucleus, but instead surrounds it. This result is confirmed in part by TEM studies on human epidermal keratinocyte (HEK) cells.

### Example 18

Cell viability studies. The viability tests of Baa and Baa-Lys(FITC)-NLS was performed by MTT colorimetric assays.

Preparation of Fullerene Peptide Stock Solution. Lyophilized Fullerene Peptides were weighed and dissolved in PBS buffer (pH=7.1), unless otherwise stated, to make a stock solution of required concentration inside a clean hood, In the case of cyclodextrin fullerene peptide complexes, equal (for H-Baa-Lys(FITC)-NLS) or triple (for H-Baa-Lys(FITC)-Lys₈) molar of γ-CD was mixed with fullerene peptides prior to solvation with the help of vertexing or sonication.

Cellular uptake and viability studies. In a typical experiment, cells were cultured in RMPI 1640 medium supplemented with 10% Fetal bovine serum (FBS, not heat inactivated) in 5% CO₂ and 37° C. humified incubator. The medium was supplemented with penicillin (100 U·mL⁻¹), streptomycin (100 ug·mL⁻¹), and glutamine (2 mM).

For experiments and microscopy, cells were seeded at 1×10⁴ cells/well on 6-well plates and grown for two days in RPMI supplemented with 10% Cells were incubated with purified fullerene peptides for 1 day in a concentration of 10 μM at 37° C. in a humidified incubator (5% CO₂). After treatment, cells were washed once with RPMI supplemented with 10% FBS and three times with phosphate buffered saline (PBS, 10 mM). Cells were visualized with an Olympus IX70 (Olympus Optical) fluorescence microscope. For nuclear staining, cells were treated with DAPI for 5 min, and then washed with PBS for three times before imaging.

Cellular proliferation studies. Cancer cells at 5×10⁴ cells/well were plated in triplicate in standard flat-bottomed 96-well tissue culture plates in the presence of fullerene peptides in PBS buffer with a final volume of 100 μL. Unless otherwise indicated, cells were grown for 48 h at 37° C. in a CO₂ incubator. Relative cell growth was determined by Cell Counting Kit-8 (CCK-8) cell proliferation assay as described by the manufacturer using an automated plate reader. Results were calculated in a blinded fashion and are the means of bi or triplicate determinations.

The following control peptides were synthesized as described hereinabove:

Phe-Lys(FITC)-NLS: Bright yellow solids, very soluble in water and methanol. Chemical formula: C₇₇H₁₁₁N₁₇O₁₅S, (Mw 1546.88 g·mol⁻¹, Exact Mass: 1545.82). The peptide was purified by preparative Varian Dynamax C18 (10 μm, 250×22.4 mm) using a linear gradient of 0.05% TFA in water and B: 0.1% TFA in acentonitrile, 5-95% in 30 min at 10 ml/min flow rate. MALDI-TOF: m/z 1547.9 [M⁺+H], 1157.6 [M⁺-FITC+H].

Lys(FITC)-Lys₈: Bright orange solids, very soluble in water and methanol Chemical formula: C₇₆H₁₂₂N₁₈O₁₅S, (Mw 1559.96 g·mol⁻¹, Exact Mass: 1558.91). The peptide was purified by preparative Varian Dynamax C18 (10 μm, 250×22.4 mm) using a linear gradient of 0.05% TFA in water and B: 0.1% TFA in acentonitrile, 5-95% in 30 min at 10 ml/min flow rate. MALDI-TOF: m/z 1559.7 [M±], 1171.1 [M⁺-FITC].

Cell viability studies were performed using standard MTT viability tests at NCSU. Samples of Baa and H-Baa-Lys(FITC)-NLS were prepared and incubated with human epidermal keratinocyte (HEK) for 24 hrs and 48 hrs respectively.

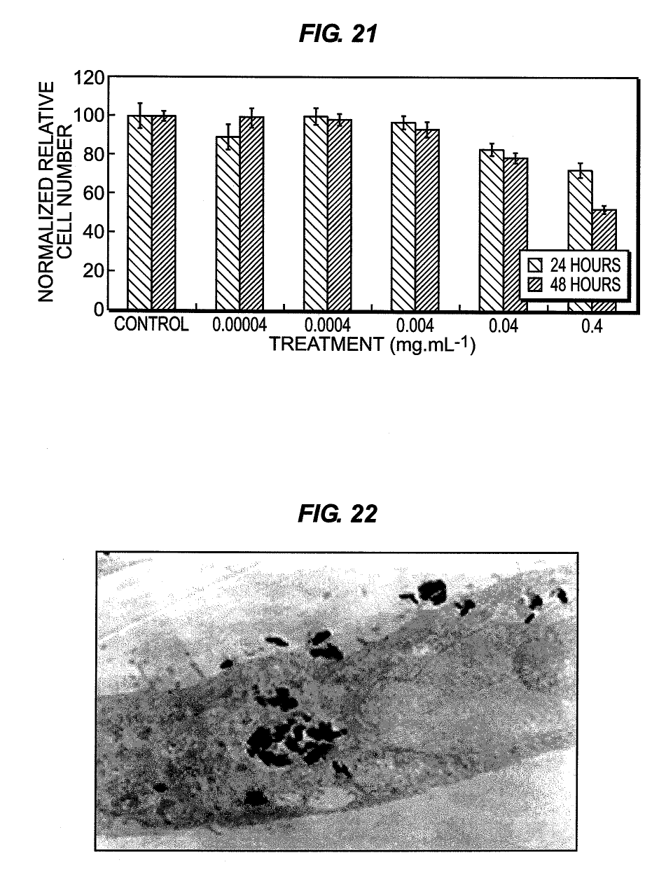

For Baa there appears to be a slight decrease in cell viability between 4.18 μM and 41.8 μM while a significant decrease occurs at 418 μM (FIG. 21). In the evaluation of cell viability of Baa, there are two factors that cannot be excluded. First, the presence of organic solvent in Baa cannot be excluded, as that is a common fact evidenced in NMR characterization. Substantial amount solvent peaks were present in the NMR spectrum even though the sample was pre-dried under high vacuum for several days. Second, the limited solubility of Baa is not higher than 0.004 mg/ml. Any concentration higher than that really gives a slurry rather than a homogeneous solution.

Given that the uptake of the Baa derivatives appears to be essentially unchanged after 24 hours it is interesting to note that the exposure time has a significant effect on the cell viability at the highest concentrations studied (FIG. 22). This would suggest that there is a secondary process in addition to uptake that causes cell death. TEM images show the presence of extensive aggregation of Baa within cells at high exposure levels (FIG. 22).

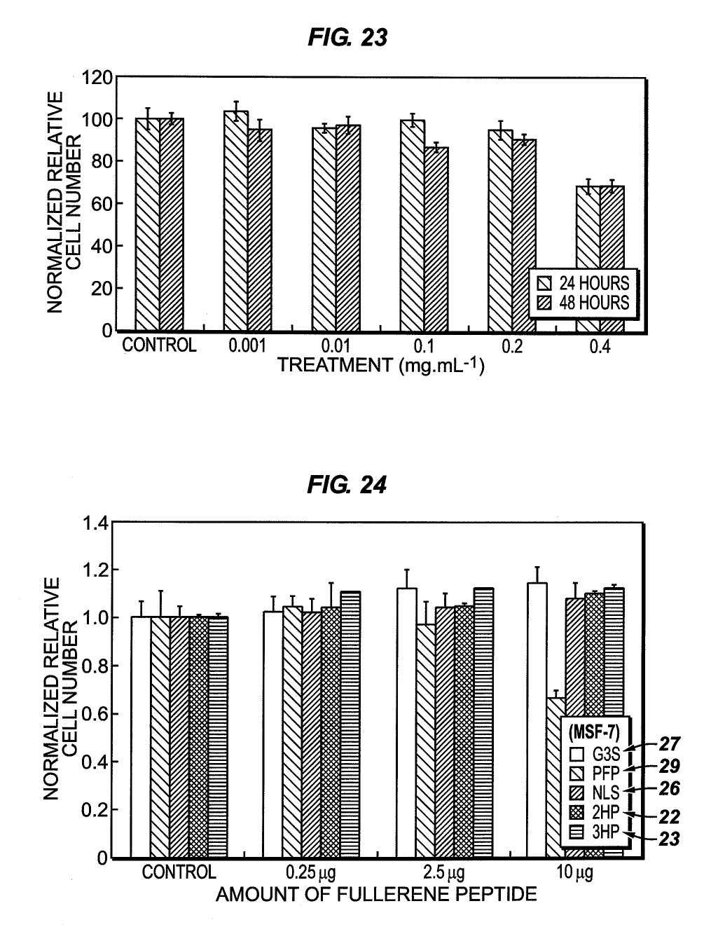

To determine the effect of the fullerene-peptide conjugate as opposed to the fullerene itself cell viability studies were performed on H-Baa-Lys(FITC)-NLS (FIG. 23). The cell viability between 86 μM (0.2 mg/mL) and 171 μM (0.4 mg/mL) is significantly decreased. A comparison with Baa shows that H-Baa-Lys(FITC)-NLS is approximately 40% of the efficacy of Baa. Thus, the effect on cell viability is not simply due to the concentration of C₆₀, but the combination the structure and cellular uptake of the peptide-fullerene conjugate.

The effect of different Baa-peptide sequences on the cell viability of MCF-7 breast cancer cells was investigated to determine if the sequence has an effect rather than the presence of the C60. Exposure of MCF-7 cells to H-Baa-Lys(FITC)-NLS (NLS) show no inhibition of cell growth up to 40 μM (FIG. 24). This is in agreement with the MTT cell viability test (see above). In a similar manner Baa-Glu-Glu-Glu-Glu-Gly-Gly-Gly-Ser-OH (G3S, 27) also shows no cellular inhibition up to 57 μM. In contrast, Baa-Arg-Gln-Ile-Lys-Ile-Trp-Phe-Gln-Asn-Arg-Arg-Met-Lys-Trp-Lys-Lys-OH (Baa-Penetratin, 29) shows a significant inhibition effect at 31.4 μM. Clearly the sequence has a significant effect on the cell viability. However, it is worth noting that this may be due to either inherent differences in toxicity or differences in cellular uptake.

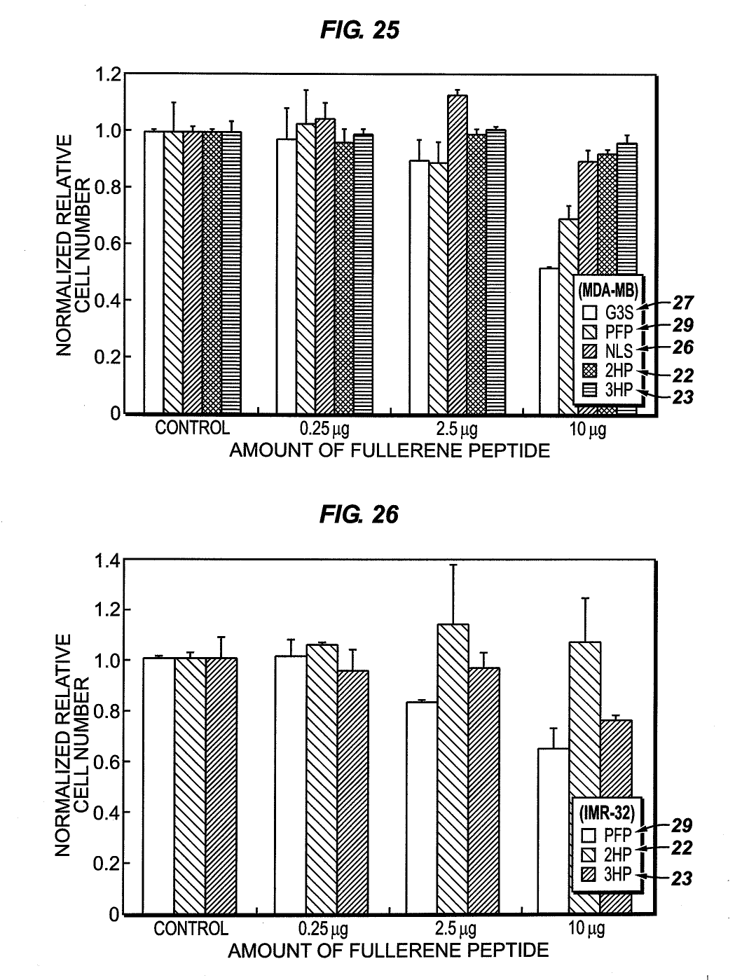

A similar comparison has been made with MDA-MB neoroblastoma cells (FIG. 25) using H-Baa-Lys(FITC)-NLS (26), Baa-Penetratin (29) and Baa-Glu-Glu-Glu-Glu-Gly-Gly-Gly-Ser-OH (27). As with the MCF-7 cells H-Baa-Lys(FITC)-NLS shows no significant inhibition, although there appears a slight effect at the highest concentration (40 μM) (FIG. 25). In contrast, both Baa-Penetratin and Baa-Glu-Glu-Glu-Glu-Gly-Gly-Gly-Ser-OH show significant inhibition for solutions above 8-14 μM. Given that both H-Baa-Lys(FITC)-NLS and Baa-Penetratin are cationic peptides which are readily taken up by the cells the cell inhibition cannot be a function of the uptake (i.e., intracellular concentration), but must also be dependent on the sequence. This is highlighted by the effect of the anionic peptide Baa-Glu-Glu-Glu-Glu-Gly-Gly-Gly-Ser-OH that has low cell uptake efficiency (compared to the other two Baa-peptides) but shows an inhibition at least as good as Baa-Penetratin.

It is well known that the lysosomes of tumor cells have a proton concentration that is 100 times (pH=5.0) lower than the physiological condition (pH=7.4). The propensity of Baa-2HP (22) and Baa-3HP (23) to precipitate from solution in acid conditions, and the known acidity within cancer cells, prompted a study of the effects of these peptides on neuroblastoma cancer cells (MDA-MB and IMR32) and breast cancer cell lines (MCF-7).

Both Baa-2HP (22) and Baa-3HP (23) shows no obvious inhibition to MCF-7 and MDA-MB). However, it shows a different trend against more flagrant IMR32 cancer cell line. Up to a concentration of 37 μM Baa-2HP shows no inhibition (FIG. 26) while Baa-3HP shows significant inhibition at a slightly lower concentration of 100 μg·mL⁻¹ (29.4 μM). This difference is possibly because the relative propensity of Baa-2HP to precipitate under acidic conditions is less than that of Baa-3HP. Thus, the formation of aggregates of Baa-3HP would cause the inhibition of cell growth. However, we cannot discount alternative factors such as the sequence and its overall charge differences. For example, the strength of the aggregate may be greater for Baa-3HP than Baa-2HP due to the stronger self-assembly forces in 3HP.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above and below described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims. It is therefore contemplated that the claims will cover any such modifications or embodiments that fall within the true scope of the invention.

What is claimed is:

1. A cell penetrating peptide for intracellular delivery comprising a fullerene modified amino acid in a peptide sequence, wherein the cell penetrating peptide is capable of crossing a cell membrane.

2. The cell penetrating peptide of claim 1, wherein the fullerene modified amino acid is a fullerene substituted phenylalanine derivative.

3. The cell penetrating peptide of claim 2, wherein the fullerene substituted phenylalanine derivative is attached to the peptide by reaction with a Boc-derivatized amino acid.

4. The cell penetrating peptide of claim 1, wherein the fullerene modified amino acid is a fullerene substituted lysine derivative.

5. The cell penetrating peptide of claim 1, wherein the fullerene modified amino acid is a fullerene substituted C₇₀ derivative.

6. The cell penetrating peptide of claim 1, wherein the fullerene modified amino acid is a metallo-fullerene derivative.

7. The cell penetrating peptide of claim 1, wherein the cell penetrating peptide is a cationic peptide.

8. The cell penetrating peptide of claim 1, wherein the cell penetrating peptide is a neutral peptide.

9. The cell penetrating peptide of claim 1, wherein the cell penetrating peptide is an anionic peptide.

10. The cell penetrating peptide of claim 1, wherein the cell penetrating peptide contains a fluorescent label.

11. The cell penetrating peptide of claim 1, wherein the cell penetrating peptide is a targeting peptide.

12. The cell penetrating peptide of claim 11, wherein the targeting peptide is a nuclear localization sequence.

13. The cell penetrating peptide of claim 1, wherein the cell penetrating peptide is delivered to the cytoplasm.

14. The cell penetrating peptide of claim 1, further comprising an entity selected from the group consisting of drug species, diagnostic probes, antigenetic peptides, peptide nucleic acids, antisense oligonucleotides, proteins, nanoparticles, liposomes and radioactive material.

15. The cell penetrating peptide of claim 14, wherein the entity is a drug species.

16. A method of intracellular delivery comprising:
(a) obtaining a cell penetrating peptide comprising a fullerene modified amino acid; and
(b) incubating the cell penetrating peptide with cells.

17. The method of claim 16, wherein the cell penetrating peptide is targeted to a specific area within the cell.

18. A method of synthesizing a cell penetrating peptide comprising a fullerene modified amino acid comprising:
(a) synthesizing a peptide using solid phase peptide synthesis; and
(b) coupling a fullerene to the peptide to form a cell penetrating fullerene peptide.

19. The method of claim 18, wherein the fullerene modified amino acid is a fullerene substituted phenylalanine derivative.

20. The method of claim 18, wherein the fullerene modified amino acid is a fullerene substituted lysine derivative.

21. The method of claim 18, wherein the fullerene modified amino acid is a C₇₀ fullerene derivative.

22. The method of claim 18, wherein the peptide is coupled using a Boc-derivatized fullerene amino acid.

23. The method of claim 18, wherein the peptide is coupled using a Fmoc-derivatized fullerene amino acid.

24. The method of claim 18, further comprising reacting the peptide with a fluorescent label.

25. A method of treatment comprising:
(a) obtaining a cell penetrating fullerene peptide; and
(b) administering to a patient a cell penetrating fullerene peptide.

26. The method of claim 25, wherein the cell penetrating fullerene peptide is designed to target a specific function of cell growth.

27. The method of claim 25, wherein the cell penetrating fullerene peptide is targeted based on the patient's DNA.

28. The method of claim 25, wherein the cell penetrating fullerene peptide delivers an entity selected from the group consisting of drug species, diagnostic probes, antigenetic peptides, peptide nucleic acids, antisense oligonucleotides, proteins, nanoparticles, liposomes and radioactive material.

29. The method of claim 28, wherein the cell penetrating fullerene peptide delivers a drug species.

30. The cell penetrating peptide of claim 2, wherein the fullerene substituted phenylalanine derivative is attached to the peptide by reaction with a Fmoc-derivatized amino acid.

31. The method of claim 18, wherein the fullerene modified amino acid is an metallo-fullerene derivative.
